(19) United States
(12) Patent Application Publication
(10) Pub. No.: US 2026/0248885 A1
(43) Pub. Date: Aug. 27, 2026

(54) S100A1 Protein for use in Treating and Preventing Infarct Extension

(71) Applicants: Patrick MOST, Heidelberg (DE); Hugo A. KATUS, Heidelberg (DE); Dorothea KEHR, Heidelberg (DE)

(72) Inventors: Patrick MOST, Heidelberg (DE); Hugo A. KATUS, Heidelberg (DE); Dorothea KEHR, Heidelberg (DE)

(21) Appl. No.: 18/871,055

(22) PCT Filed: Jun. 1, 2023
(86) PCT No.: PCT/EP2023/064763

§ 371 Date: Dec. 2, 2024

(30) Foreign Application Priority Data
Jun. 2, 2022 (EP) 22177107.4

(51) Int. Cl. A61K 38/17; A61K 48/00; A61P 9/10

(52) U.S. Cl. CPC A61K 38/1738; A61K 48/0033; A61K 48/0058; A61P 9/10

(57) ABSTRACT

The present invention pertains to an S100A1 protein, a biologically active fragment or variant thereof, to a nucleic acid encoding said S100A1 protein or said biologically active fragment or variant thereof, to a vector comprising said nucleic acid, and to a pharmaceutical composition comprising said protein or biologically active fragment or variant thereof, the nucleic acid and/or the vector, for use in treating and/or preventing infarct extension in a patient in need thereof.

The present invention relates to the S100A1 protein and nucleic acids encoding said protein for use in treating and/or preventing myocardial infarct extension in a patient in need thereof, as well as to vectors comprising said nucleic acid and to respective pharmaceutical compositions.

### BACKGROUND OF THE INVENTION

Muscle tissue is subdivided into skeletal muscle, cardiac muscle, and smooth muscle tissue and can be considered the biggest organ of a vertebrate. Skeletal muscle and cardiac muscle belong to the striated muscle tissue and share many functional aspects.

Ischemia of the heart muscle due to various causes, e.g., an occlusion of a coronary arterial vessel, leads to muscle necrosis with subsequent sterile inflammation and death of the myocardium. Many studies have shown that long-term prognosis after such a myocardial infarction is closely related to myocardial function, which is determined primarily by the size and location of the infarct.

Two complications occurring early in the post-infarct period that can significantly increase the functional infarct size are known: infarct extension and infarct expansion. While myocardial extension is characterized by an increase of the mass of necrotic tissue, myocardial expansion is associated with thinning and dilating the infarcted zone. Both, infarct extension and infarct expansion have been associated with increased mortality and morbidity both early and late after myocardial infarction (Hochman & Bulkley, “Myocardial Infarct Expansion and Extension” in: Califf, R. M., Wagner, G. S. (eds) “Acute Coronary Care”. 1985 Springer, Boston, MA.). In infarct extension, the infarcted muscle tissue is ultimately replaced by a fibrotic scar due to secondary cardiomyocyte necrosis (M. B. Ratcliffe, “Non-Ischemic Infarct extension: a new type of infarct enlargement and a potential therapeutic target”, Journal of the American College of Cardiology, 40, 2002). By this, the growing scar continuously consumes border zone myocardium and weakens the contractile performance of the heart. Together with a persistent activity of both the innate and adaptive immune system in the hemodynamically stressed myocardium, the border zone of the infarcted area is thereby exposed to chronic hemodynamic overload and a sustained proinflammatory environment with cytotoxic cytokines and chemokines that leads to continuing secondary death of cardiomyocytes. Respective processes include, but are not limited to, e.g., post-myocardial infarction (post-MI) neutrophil granulocyte and macrophage infiltration and persistence as well as activation and persistence of B- and T-cell based immune responses in the heart, encompassing the release of various interleukins, interferons, cytokines and chemokines in the post-MI stressed heart. Overall, this entails the extension of the myocardial infarct zone in contrast to myocardial infarct expansion, which is distinguished as thinning of the scar tissue as a result of progressive ventricular dilatation (J. Wenk et al., “First Evidence of Depressed Contractility in the Border Zone of Human Myocardial Infarction”, Ann Thorac Surg., 93, 2012; S. Bansal et al., “Activated T Lymphocytes are Essential Drivers of Pathological Remodeling in Ischemic Heart failure”, Circ Heart Failure, 10, 2017; G. Garcia-Rivas et al., The role of B cells in heart failure and implications for future immunomodulatory treatment strategies”, 7, 2020; P. Westman et al., “Inflammation as a driver of adverse left ventricular remodeling after acute myocardial infarction”, Journal of the American College of Cardiology, 67, 2017).

Albeit a number of treatments are available for treating myocardial infarction, at present, there are no clinical therapies available to inhibit the post-MI processes and thereby limit collateral damage to non-infarcted myocardium due to infarct extension (M. B. Ratcliffe, “Non-Ischemic Infarct extension: a new type of infarct enlargement and a potential therapeutic target”, Journal of the American College of Cardiology, 40, 2002).

### SUMMARY OF THE INVENTION

In view of the lack of suitable means and methods for preventing or treating myocardial infarct extension, the present invention provides according to a first aspect, a S100A1 protein, a biologically active fragment or variant thereof or a nucleic acid encoding said S100A1 protein or the biologically active fragment or variant thereof, for use in treating and/or preventing infarct extension in a patient in need thereof, wherein the fragment or variant has at least 80% sequence identity to SEQ ID NO:1.

In a second aspect, the present invention provides a vector comprising a nucleic acid encoding an S100A1 protein or a biologically active fragment or variant thereof having at least 80% sequence identity to SEQ ID NO:1, for use in treating and/or preventing infarct extension in a patient in need thereof.

In a third aspect, the present invention provides a pharmaceutical composition comprising:
an S100A1 protein, a biologically active fragment or variant thereof having at least 80% sequence identity to SEQ ID NO:1 or a nucleic acid encoding said S100A1 protein, fragment or variant; (ii) a vector comprising a nucleic acid encoding an S100A1 protein or a biologically active fragment or variant thereof having at least 80% sequence identity to SEQ ID NO:1; or (iii) a pharmaceutically acceptable salt of (i) or (ii), for use in treating and/or preventing infarct extension in a patient in need thereof. The pharmaceutical composition optionally further comprises a pharmaceutically acceptable excipient, carrier, and/or diluent.

According to one embodiment, the S100A1 protein, the biologically active fragment or variant thereof, the nucleic acid, the vector or the pharmaceutical composition for use according to one of claims 1 to 3, wherein the S100A1 protein has the sequence as set forth in SEQ ID NO:1.

According to a further embodiment, the vector is selected from the group consisting of plasmid vectors, cosmid vectors, phage vectors such as lambda phage, filamentous phage vectors, viral vectors, viral like particles, and bacterial spores.

According to a preferred embodiment, the viral vector is selected from the group consisting of an adenoviral vector, adeno-associated viral (AAV) vector, alphaviral vector, herpes viral vector, measles viral vector, pox viral vector, vesicular stomatitis viral vector, retroviral vector and lentiviral vector. More preferably, the adeno-associated viral (AAV) vector is selected from the group consisting of AAV5, AAV6, and AAV9, preferably AAV5.

According to a further embodiment, the expression of the S100A1 protein is controlled by a heart tissue specific promoter. Preferably, the heart-tissue specific promoter is selected from the group consisting of Cardiac Actin Enhancer/Elongation Factor 1 promoter Cytomegalo-virus enhancer/Myosin light chain ventricle 2 promoter and Troponin.

According to an embodiment, the protein, the biologically active fragment thereof, the nucleic acid, the vector or the pharmaceutical composition is administered via the oral, intravenous, intramucosal, intraarterial, intramuscular or intracoronal route.

According to a further embodiment, the protein, the biologically active fragment or variant thereof, the nucleic acid, the vector or the pharmaceutical composition is administered via the intravenous route. Preferably, the protein, the nucleic acid, the vector or the pharmaceutical composition is administered retrograde into the coronary venous system of the patient.

According to a preferred embodiment, the S100A1 protein, the biologically active fragment or variant thereof, the nucleic acid, the vector or the pharmaceutical composition is administered between 1 day and four weeks after a myocardial infarction in said patient has become post-ischemic.

According to an embodiment, the intracellular level of the S100A1 protein is raised for a period of at least 7 days.

According to a further embodiment, the vector is administered in a single dose of 1×10¹³ vector genomic copies (vgc).

In a fourth aspect, the present invention relates to a method for treating and/or preventing infarct extension in a patient in need thereof. The method comprises administering to the patient:
(i) an S100A1 protein, a biologically active fragment or variant thereof having at least 80% sequence identity to SEQ ID NO:1, or a nucleic acid encoding said S100A1 protein or said fragment thereof,
(ii) a vector comprising a nucleic acid encoding an S100A1 protein or a biologically active fragment or variant thereof having at least 80% sequence identity to SEQ ID NO:1, or
(iii) a pharmaceutically acceptable salt of (i) or (ii).

According to an embodiment, the S100A1 protein has the sequence as set forth in SEQ ID NO: 1.

According to a preferred embodiment, the vector is selected from the group consisting of plasmid vectors, cosmid vectors, phage vectors such as lambda phage, filamentous phage vectors, viral vectors, viral like particles, and bacterial spores. According to a preferred embodiment, the viral vector is selected from the group consisting of an adenoviral vector, adeno-associated viral (AAV) vector, alphaviral vector, herpes viral vector, measles viral vector, pox viral vector, vesicular stomatitis viral vector, retroviral vector and lentiviral vector. According to a specifically preferred embodiment, the adeno-associated viral (AAV) vector is selected from the group consisting of AAV5, AAV6, and AAV9.

According to an embodiment, the expression of the S100A1 protein is controlled by a heart tissue specific promoter. According to a preferred embodiment, the heart-tissue specific promoter is selected from the group consisting of Cardiac Actin Enhancer/Elongation Factor 1 promoter Cytomegalo-virus enhancer/Myosin light chain ventricle 2 promoter and Troponin.

According to a further embodiment, the protein, the biologically active fragment thereof, the nucleic acid, the vector or the pharmaceutical composition is administered via the oral, intravenous, intramucosal, intraarterial, intramuscular or intracoronal route. According to a preferred embodiment, the protein, the biologically active fragment thereof, the nucleic acid, the vector or the pharmaceutical composition is administered via the intravenous route. Preferably, the protein, the nucleic acid, the vector or the pharmaceutical composition is administered retrograde into the coronary venous system of the patient.

According to a preferred embodiment, the protein, the biologically active fragment thereof, the vector or the pharmaceutical composition is administered between 1 day and four weeks after a myocardial infarction in said patient has become post-ischemic.

According to a further embodiment, the intracellular level of the S100A1 protein is raised for a period of at least 7 days.

According to an embodiment, the vector is administered in a single dose of 1×10¹³ vector genomic copies (vgc).

Other features and advantages of the present invention will be apparent from the following detailed description and the claims.

### LIST OF FIGURES

FIG. 1: A and B show representative sequential fluoroscopic images as part of the cardiac-targeted retrograde intravenous gene delivery (CRID) standardized operational procedure (SOP) as previously published (Pleger et al., “Cardiac AAV9-S100A1 gene therapy rescues post-ischemic heart failure in a preclinical large animal model”, Science Translational Medicine, 3, 2011). A documents the appropriate position both of the retroinfusion catheter (arrow; 1) in the cardiac anterior interventricular vein (AIV) of the porcine heart and of the balloon catheter (arrow; 2) in the left anterior descending artery (balloon inflated at the time of the image) via the guiding catheter. B depicts the retrograde installation of contrast agent in the AIV (arrow; 3) via the retroperfusion catheter to ascertain the correct position and sufficient blocking of the retroperfusion catheter tip prior to CRID. C and D illustrate the luc reporter gene activity along the apico-basal axis of the healthy porcine heart 4 weeks after CRID of rAAV5-luc compared with AAV6-luc and rAAV9-luc. C shows that rAAV5 mediates significantly higher luc activity levels than rAAV9 in each of the three myocardial segments. D highlights that AAV5 results in a significantly lower but less heterogenous myocardial distribution of the reporter gene activity than rAAV6 across the the apico-basal cardiac axis. Luc reporter gene activity is given as relative light units (RLU) in relation to mg of extracted tissue protein. ****P<0.001; ***P<0.01; **P<0.03; n=5 animals per group. E and F display the absolute and relative extra-cardiac reporter gene activity levels for each rAAV serotype 4 weeks after the CRID SOP. E reveals that rAAV6 generated the highest off-target luc reporter gene activity across the tested organs when compared to either rAAV5 or rAAV9. F unveils that rAAV9 exerts by far the highest off-target reporter gene activity across the tested organs when compared to either rAAV5 or rAAV9. Luc reporter gene activity is given as relative light units (RLU) in relation to mg of extracted tissue protein. ****P<0.001; ***P<0.01; **P<0.03, *P<0.05.

FIG. 2: A describes the design of the rAAV5-hs100a1 pig study using the previously established MI, CMR and CRID SOPs (P. Most et al., “Cardiac AAV9-S100A1 gene therapy rescues post-ischemic heart failure in a preclinical large animal model”, Science Translational Medicine, 3, 2011; P. Raake et al.; “Comprehensive cardiac phenotyping in large animals: comparison of pressure volume analysis and cardiac magnetic resonance imaging in pig post-myocardial infarction systolic heart failure”, Int J Cardiovasc Imaging, 35, 2019). At day 15 post-MI, only pigs that exceeded a LV-MI size >14% in the CMR assessment were randomized to either CRID of rAAV5-hs100a1 (hs100a1) or control vector treatment. 3-months after gene therapy (GT), the two study endpoints LV-EF % and LV-MI mass were assessed by CMR SOP. *Procedures were conducted by experimenters blinded to the type of treatment. B highlights the significant enlargement of the absolute infarcted LV-MI mass in the control vector treated group versus the hs100a1 intervention. The extension of LV-MI mass was completely absent in the hs100a1 treated group. Data are given as individual change before and after the 3-month GT period (post-GT change). **P<0.03; control n=4 and hs100a1 n=5. C confirms the finding of the hs100a1-mediated protection against LV infarct extension by post-GT assessment of the global LV T1 relaxation time (ms). The post-GT Tl relaxation time difference tends to decrease in the hs100a1-treated group. Data are given as individual change per animal before and after the 3-month GT period (post-GT change). *P<0.05; control n=4 and hs100a1 n=4. D depicts the significant relative post-GT improvement of LV-EF % in the hs100a1 versus the control treated group. Data are given as individual difference per animal 3-months after GT (post-GT). **P<0.03; n=4 and hs100a1 n=4.

FIG. 3: A demonstrates that a systemic assessment of the vector distribution (black encircled box) was conducted from biosamples of each animal 3-months after rAAV5-hs100a1 gene therapy. B shows an equal transduction of the porcine heart by the control rAAV5 (n=3) and rAAV5-hs100a1 vector (n=4) 3-months post-CRID SOP. Data are shown as vector genome copy (vgc) numbers per ug of isolated genomic DNA (gDNA) from the porcine heart for each group. C displays the systemic distribution of the rAAV5-hs100a1 vector (n=4) 3-months post-CRID in a representative set of organs, including the heart, liver, lung, kidney and brain. Data are presented as individual organ/heart ratio of the respective organ rAAV5-hs100a1 vector genome copy (vgc) number/μg genomic DNA. D highlights the cardiac-contained expression of hs100a1 mRNA. The cardiac-biased promoter effectively suppresses extra-cardiac transcriptional activity of rAAV5. *P<0.05. Data are given as the ratio for hs100a1 mRNA copy numbers per rAAV5-hs100a1 vgc of for each organ normalized to the heart value. hs100a1 mRNA tissue copy numbers were calibrated using an hs100a1 gene-containing plasmid standard curve.

FIG. 4: A demonstrates that bulk myocardial tissue RNAseq and subsequent a WGCNA (black encircled box) was conducted 3-months after rAAV5-hs100a1 gene therapy. B shows the result of the WGCNA for the two clinical endpoints LV-EF % and MI extension of the rAAV5-hs100a1 pig study. Only module eigengenes (MEs) with a strong and significant negative correlation either with LV-EF % or MI extension and annotated pathways by REACTOME, GEO or KEGG pathway analysis were considered. ME turquoise and ME cyan strongly correlate with LV-EF % (P<0.001) and MI extension (P<0.03), respectively. C highlights the annotated and significantly enriched pathways in the ME turquoise that negatively correlates with LV-EF %. Pathway annotation for the significantly correlated gene ensembles was successful retrieved from REACTOME. The mostly proinflammatory gene ensembles, which negatively correlate with LF-EF % changes reflect activation of the innate immune system (e.g., neutrophil degranulation) and the adaptive immune system (e.g., T-cell receptor signaling and signaling by the B-cell receptor) in the post-MI pig heart, display a lower expression in the rAAV5-hs100a1 group compared with control since the respective rAAV5-hs100a1/control n-fold RNAseq gene expression ratios were <1). D and E show the annotated and significantly enriched pathways in the ME cyan that negatively correlates with MI extension. Pathway annotation for the significantly correlated gene ensembles was successful with REACTOME (D) and KEGG (E). The mostly energy performance (e.g., mitochondrial translation), cardioprotective (e.g., signaling by retinoic acid and FGRC2c activation) and calcium signaling pathway gene ensembles, which negatively correlate with MI extension, display a higher expression in the rAAV5-hs100a1 group compared with control since the respective rAAV5-hs100a1/control n-fold RNAseq. gene expression ratio was >1).

FIG. 5: A describes the design of the confirmatory rAAV5-hs100a1 mouse study using intramyocardial injections immediately after experimental MI. 4-weeks after gene therapy, both LV-EF % and inflammatory gene expression were determined by echocardiography and RT-PCR (black encircled box). B shows the published method by which intramyocardial injections and MI were conducted (Most et al. “Cardiac S100A1 protein levels determine contractile performance and propensity toward heart failure after myocardial infarction”, 114, 2006). C displays the expected improvement of LV-EF % by rAAV5-hs100a1 compared to control (rAAV5-gfp). D confirms the mitigation of MI size by rAAV5-hs100a1 vs. control. n=7 animals per group. E unveils the attenuation of inflammatory cell marker abundance in the rAAV5-hs100a1 treated post-MI myocardium compared to control. F highlights the mitigation of inflammatory cytokine expression in the rAAV5-hs100a1 treated post-MI myocardium compared to control. ****P<0.001; ***P<0.01; **P<0.03, *P<0.05; n=4 animals per group.

SEQUENCES

SEQ ID NO: 1
MGSELETAMETLINVFHAHSGKEGDKYKLSKKELKELLQTELSGFLDAQ
KDVDAVDKVMKELDENGDGEVDFQEYVVLVAALTVACNNFFWENS

### DETAILED DESCRIPTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

To practice the present invention, unless otherwise indicated, conventional methods of chemistry, biochemistry, and recombinant DNA techniques are employed which are explained in the literature in the field (cf., e.g., Molecular Cloning: A Laboratory Manual, 2^{nd} Edition, J. Sambrook et al. eds., Cold Spring Harbor Laboratory Press, Cold Spring Harbor 1989). In the following, the elements of the present invention will be described. These elements are listed with specific embodiments; however, it should be understood that they may be combined in any manner and in any number to create additional embodiments. The various described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

To practice the present invention, unless otherwise indicated, conventional methods of chemistry, biochemistry, cell biology, and recombinant DNA techniques are employed which are explained in the literature in the field (cf., e.g., Molecular Cloning: A Laboratory Manual, 2^{nd} Edition, J. Sambrook et al. eds., Cold Spring Harbor Laboratory Press, Cold Spring Harbor 1989). Furthermore, conventional methods of clinical cardiology are employed which are also explained in the literature in the field (cf., e.g., Practical Methods in Cardiovascular Research, S. Dhein et al. eds., Springer Verlag Berlin Heidelberg, 2005).

Throughout this specification and the claims which follow, unless the context requires otherwise, the word “comprise”, and variations such as “comprises” and “comprising” will be understood to imply the inclusion of a stated integer or step or group of integers or steps but not the exclusion of any other integer or step or group of integers or steps. As used in this specification and the appended claims, the singular forms “a”, “an”, and “the” include plural referents, unless the content clearly dictates otherwise. The term “about” when used in connection with a numerical value is meant to encompass numerical values within a range having a lower limit that is 5% smaller than the indicated numerical value and having an upper limit that is 5% larger than the indicated numerical value.

As used herein, the term “and/or” means that it refers to either one or both/all of the options cited in the context of this term. For example, when referring to “treating and/or preventing myocardial infarct extension”, the term is to be interpreted to mean: 1) treating myocardial infarct extension; 2) preventing myocardial infarct extension; or 3) treating and preventing myocardial infarct extension.

In the context of the different aspects of present invention, the term “peptide” refers to a short polymer of amino acids linked by peptide bonds. It has the same chemical (peptide) bonds as proteins, but is commonly shorter in length.

In the context of the different aspects of present invention, the term “polypeptide” refers to a single linear chain of amino acids bonded together by peptide bonds. A polypeptide can be one chain of a protein that is composed of more than one chain or it can be the protein itself if the protein is composed of one chain.

In the context of the different aspects of present invention, the term “protein” refers to a molecule comprising multiple amino acid residues and/or one or more polypeptides that resume a secondary and tertiary structure and additionally refers to a protein that is made up of several polypeptides, i.e. several subunits, forming quaternary structures. The protein has sometimes non-peptide groups attached, which can be called prosthetic groups or cofactors.

The terms “polynucleotide” and “nucleic acid” are used synonymously and are understood as single or double-stranded oligo- or polymers of deoxyribonucleotide or ribonucleotide bases or both. The depiction of a single strand of a nucleic acid also defines (at least partially) the sequence of the complementary strand. The nucleic acid may be single or double stranded, or may contain portions of both double and single stranded sequences. In the context of the different aspects of present invention, the term nucleic acid comprises cDNA, genomic DNA, recombinant DNA, CRNA and mRNA. A nucleic acid may consist of an entire gene, or a portion thereof, the nucleic acid may also be a microRNA (miRNA) or small interfering RNA (siRNA). The term “oligonucleotide” when used in the context of one of the different aspects of present invention, refers to a nucleic acid of up to about 50 nucleotides, e.g. 2 to about 50 nucleotides in length. “Nucleic acid” molecules are understood as a polymeric or oligomeric macromolecule made from nucleotide monomers. Nucleotide monomers are composed of a nucleobase, a five-carbon sugar (such as but not limited to ribose or 2′-deoxyribose), and one to three phosphate groups. Typically, a polynucleotide is formed through phosphodiester bonds between the individual nucleotide monomers. In the context of the present invention referred to nucleic acid molecules include but are not limited to ribonucleic acid (RNA), deoxyribonucleic acid (DNA), and mixtures thereof such as e.g. RNA-DNA hybrids. The nucleic acid may be obtained by biological, biochemical or chemical synthesis methods or any of the methods known in the art. The nucleic acids, can e.g. be synthesized chemically, e.g. in accordance with the phosphotriester method (see, for example, Uhlmann, E. & Peyman, A. (1990) Chemical Reviews, 90, 543-584). “Aptamers” are nucleic acids which bind with high affinity to a polypeptide. Aptamers can be isolated by selection methods from a large pool of different single-stranded RNA molecules (see e.g. Jayasena (1999) Clin. Chem., 45, 1628-50; Klug and Famulok (1994) M. Mol. Biol. Rep., 20, 97-107; U.S. Pat. No. 5,582,981). Aptamers can also be synthesized and selected in their mirror-image form, for example as the L-ribonucleotide (Nolte et al. (1996) Nat. Biotechnol., 14, 1116-9; Klussmann et al. (1996) Nat. Biotechnol., 14, 1112-5). Forms which have been isolated in this way enjoy the advantage that they are not degraded by naturally occurring ribonucleases and, therefore, possess greater stability. Nucleic acids may be degraded by endonucleases or exonucleases, in particular by DNases and RNases which can be found in the cell. It is, therefore, advantageous to modify the nucleic acids in order to stabilize them against degradation, thereby ensuring that a high concentration of the nucleic acid is maintained in the cell over a long period of time (Beigelman et al. (1995) Nucleic Acids Res. 23:3989-94). Typically, such stabilization can be obtained by introducing one or more internucleotide phosphorus groups or by introducing one or more non-phosphorus internucleotides. Suitable modified internucleotides are compiled in Uhlmann and Peyman (1990), supra (see also Beigelman et al. (1995) Nucleic Acids Res. 23:3989-94). Modified internucleotide phosphate radicals and/or non-phosphorus bridges in a nucleic acid which can be employed in one of the uses according to the invention contain, for example, methyl phosphonate, phosphorothioate, phosphoramidate, phosphorodithioate and/or phosphate esters, whereas non-phosphorus internucleotide analogues contain, for example, siloxane bridges, carbonate bridges, carboxymethyl esters, acetamidate bridges and/or thioether bridges. It is also the intention that this modification should improve the durability of a pharmaceutical composition which can be employed in one of the uses according to the invention.

As used herein, the term “vector” refers to a protein or a polynucleotide or a mixture thereof which is capable of being introduced or of introducing the proteins and/or nucleic acid comprised therein into a cell. In the context of the present invention, it is preferred that the genes of interest encoded by the introduced polynucleotide are expressed within the cell upon introduction of the vector or vectors. Examples of suitable vectors include but are not limited to plasmids, cosmids, phages, viruses or artificial chromosomes.

As used herein, the term “variant” is to be understood as a polynucleotide or protein which differs in comparison to the polynucleotide or protein from which it is derived by one or more changes in its length or sequence. The polypeptide or polynucleotide from which a protein or nucleic acid variant is derived is also known as the parent polypeptide or polynucleotide. The term “variant” comprises “fragments” or “derivatives” of the parent molecule. Typically, “fragments” are smaller in length or size than the parent molecule, whilst “derivatives” exhibit one or more differences in their sequence in comparison to the parent molecule. According to the present invention, a fragment of SEQ ID NO:1 has a minimum length of 9 amino acids. A preferred fragment of SEQ ID NO: 1 has a length of between 10 and 30 amino acids, such as 10, 11, 12, 13, 14, 15, 16, 17, 18, 19, 20, 21, 22, 23, 24, 25, 26, 27, 28, 29 or 30 amino acids. A particularly preferred fragment of SEQ ID NO: 1 has 20 amino acids.

Also encompassed are modified molecules such as but not limited to post-translationally modified proteins (e.g., glycosylated, biotinylated, phosphorylated, ubiquitinated, palmitoylated, or proteolytically cleaved proteins) and modified nucleic acids such as methylated DNA. Also, mixtures of different molecules such as but not limited to RNA-DNA hybrids. Typically, a variant is constructed artificially, preferably by gene-technological means whilst the parent polypeptide or polynucleotide is a wild-type protein or polynucleotide. However, also naturally occurring variants are to be understood to be encompassed by the term “variant” as used herein. Further, the variants usable in the present invention may also be derived from homologs, orthologs, or paralogs of the parent molecule or from artificially constructed variant, provided that the variant exhibits at least one biological activity of the parent molecule, e.g., is functionally active.

As used herein, the term protein “variant” is to be understood as a polypeptide which differs in comparison to the polypeptide from which it is derived by one or more changes in the amino acid sequence. The polypeptide from which a protein variant is derived is also known as the parent polypeptide. Typically, a variant is constructed artificially, preferably by gene-technological means. Typically, the parent polypeptide is a wild-type protein or wild-type protein domain. In the context of the present invention, it is further preferred that a parent polypeptide is the consensus sequence of two or more wild-type polypeptides. Further, the variants usable in the present invention may also be derived from homologs, orthologs, or paralogs of the parent polypeptide or from artificially constructed variant, provided that the variant exhibits at least one biological activity of the parent polypeptide. The changes in the amino acid sequence may be amino acid exchanges, insertions, deletions, N-terminal truncations, or C-terminal truncations, or any combination of these changes, which may occur at one or several sites. In preferred embodiments, a variant usable in the present invention exhibits a total number of up to 20 (up to 1, 2, 3, 4, 5, 6, 7, 8, 9, 10, 11, 12, 13, 14, 15, 16, 17, 18, 19 or 20) changes in the amino acid sequence (e.g., exchanges, insertions, deletions, N-terminal truncations, and/or C-terminal truncations). The amino acid exchanges may be conservative and/or non-conservative.

Semi-conservative and especially conservative amino acid substitutions, wherein an amino acid is substituted with a chemically related amino acid are preferred. Typical substitutions are among the aliphatic amino acids, among the amino acids having aliphatic hydroxyl side chain, among the amino acids having acidic residues, among the amide derivatives, among the amino acids with basic residues, or the amino acids having aromatic residues. Typical semi-conservative and conservative substitutions are:

| | Conservative | Semi-conservative |
|---|---|---|
| Amino acid | substitution | substitution |
| A | G; S; T | N; V; C |
| C | A; V; L | M; I; F; G |
| D | E; N; Q | A; S; T; K; R; H |
| E | D; Q; N | A; S; T; K; R; H |
| F | W; Y; L; M; H | I; V; A |
| G | A | S; N; T; D; E; N; Q |
| H | Y; F; K; R | L; M; A |
| I | V; L; M; A | F; Y; W; G |
| K | R; H | D; E; N; Q; S; T; A |
| L | M; I; V; A | F; Y; W; H; C |
| M | L; I; V; A | F; Y; W; C; |
| N | Q | D; E; S; T; A; G; K; R |
| P | V; I | L; A; M; W; Y; S; T; C; F |
| Q | N | D; E; A; S; T; L; M; K; R |
| R | K; H | N; Q; S; T; D; E; A |
| S | A; T; G; N | D; E; R; K |
| T | A; S; G; N; V | D; E; R; K; I |
| V | A; L; I | M; T; C; N |
| W | F; Y; H | L; M; I; V; C |
| Y | F; W; H | L; M; I; V; C |

Changing from A, F, H, I, L, M, P, V, W or Y to C is semi-conservative if the new cysteine remains as a free thiol. Furthermore, the skilled person will appreciate that glycines at sterically demanding positions should not be substituted and that P should not be introduced into parts of the protein which have an alpha-helical or a beta-sheet structure.

Alternatively or additionally, a “variant” as used herein, can be characterized by a certain degree of sequence identity to the parent polypeptide or parent polynucleotide from which it is derived. More precisely, a protein variant in the context of the present invention exhibits at least 80% sequence identity to its parent polypeptide. A polynucleotide variant in the context of the present invention exhibits at least 80% sequence identity to SEQ ID NO:1. According to a preferred embodiment, the parent polypeptide is SEQ ID NO:1.

The term “at least 80% sequence identity” is used throughout the specification with regard to polypeptide and polynucleotide sequence comparisons. This expression preferably refers to a sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to SEQ ID NO:1.

Variants may additionally or alternatively comprise deletions of amino acids, which may be N-terminal truncations, C-terminal truncations or internal deletions or any combination of these. Such variants comprising N-terminal truncations, C-terminal truncations and/or internal deletions are referred to as “deletion variants” in the context of the present application.

Fragments may be naturally occurring (e.g. splice variants) or it may be constructed artificially, preferably by gene-technological means. According to the present invention, a fragment comprises at least 9 amino acids of SEQ ID NO: 1. Preferably, a fragment or deletion variant has a deletion of up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or 85 amino acids compared to SEQ ID NO:1, preferably at its N-terminus and/or internally as compared to the parent polypeptide of SEQ ID NO: 1, more preferably at its N-terminus. A particularly preferred fragment according to the present invention is a peptide consisting of amino acids Y75 to S94 of SEQ ID NO:1. In cases where a fragment sequence is compared with SEQ ID NO: 1, the sequence identity percentage is to be calculated with reference to the fragment, i.e. the shorter of the two sequences to be compared. The sequence identity is then determined on the basis of the overlap between the fragment and SEQ ID NO:1. The percentage of sequence identity is preferably determined via sequence alignments. Such alignments can be carried out with several art-known algorithms. For example, the grade of sequence identity (sequence matching) may be calculated using e.g. BLAST (blastp) or EMBOSS Needle (EMBL-European Bioinformatics Institute). A respective algorithm is incorporated into the BLASTN and BLASTP programs of Altschul et al. (1990) J. Mol. Biol. 215:403-410, and in Altschul et al. (1997) Nucleic Acids Res. 25:3389-3402.

Fragments of SEQ ID NO:1 that additionally comprise one or more amino acid substitutions within their amino acid sequence compared to SEQ ID NO:1 are also referred to as “deletion variants”.

Additionally or alternatively a deletion variant may occur not due to structural deletions of the respective amino acids as described above, but due to these amino acids being inhibited or otherwise not able to fulfill their biological function. Typically, such functional deletion occurs due to the insertions to or exchanges in the amino acid sequence that changes the functional properties of the resultant protein, such as but not limited to alterations in the chemical properties of the resultant protein (i.e. exchange of hydrophobic amino acids to hydrophilic amino acids), alterations in the post-translational modifications of the resultant protein (e.g. post-translational cleavage or glycosylation pattern), or alterations in the secondary or tertiary protein structure. Additionally or alternatively, a functional deletion may also occur due to transcriptional or post-transcriptional gene silencing (e.g. via siRNA) or the presence or absence of inhibitory molecules such as but not limited to protein inhibitors or inhibitory antibodies.

In the context of the present invention it is preferred that a protein or polypeptide being “functionally deleted” refers to the fact that the amino acids or nucleotides of the corresponding sequence are either deleted or present but not fulfilling their biological function.

As used herein, an “individual” means any mammal, reptile or bird that may benefit from the present invention. Preferably, an individual is selected from the group consisting of laboratory animals (e.g. mouse, rat or rabbit), domestic animals (including e.g. guinea pig, rabbit, horse, donkey, cow, sheep, goat, pig, chicken, duck, camel, cat, dog, turtle, tortoise, snake, or lizard), or primates including chimpanzees, bonobos, gorillas and human beings. It is particularly preferred that the “individual” is a human being.

The term “patient” as used herein refers to any mammal, reptile or bird that may benefit from a prognosis, diagnosis, identification or treatment of a disease or disorder. Preferably, a “patient” is selected from the group consisting of laboratory animals (e.g. mouse or rat), domestic animals (including e.g. guinea pig, rabbit, horse, donkey, cow, sheep, goat, pig, chicken, camel, cat, dog, turtle, tortoise, snake, or lizard), or primates including chimpanzees, bonobos, gorillas and human beings. It is particularly preferred that the “patient” is a human being.

The terms “disease” and “disorder” are used interchangeably herein, referring to an abnormal condition, especially an abnormal medical condition such as an illness or injury, wherein a tissue, an organ or an individual is not able to efficiently fulfil its function anymore. Typically, but not necessarily, a disease is associated with specific symptoms or signs indicating the presence of such disease. The presence of such symptoms or signs may thus, be indicative for a tissue, an organ or an individual suffering from a disease. An alteration of these symptoms or signs may be indicative for the progression of such a disease. A progression of a disease is typically characterised by an increase or decrease of such symptoms or signs which may indicate a “worsening” or “bettering” of the disease. The “worsening” of a disease is characterised by a decreasing ability of a tissue, organ or organism to fulfil its function efficiently, whereas the “bettering” of a disease is typically characterised by an increase in the ability of a tissue, an organ or an individual to fulfil its function efficiently. A tissue, an organ or an individual being at “risk of developing” a disease is in a healthy state but shows potential of a disease emerging. Typically, the risk of developing a disease is associated with early or weak signs or symptoms of such disease. In such case, the onset of the disease may still be prevented by treatment. Examples of a disease include but are not limited to traumatic diseases, inflammatory diseases, infectious diseases, and cardiac disorders. Examples of cardiac disorders include but are not limited to postischemic contractile dysfunction, congestive heart failure, cardiogenic shock, septic shock, primary or secondary cardiomyopathy, dysfunction of heart valves, ventricular disorder, and preferably myocardial infarction. Primary cardiomyopathy includes inherited cardiomyopathy and cardiomyopathy caused by spontaneous mutations. The secondary cardiomyopathy includes ischemic cardiomyopathy caused by arteriosclerosis, dilated cardiomyopathy caused by infection or intoxication of the myocard, hypertensive heart disease caused by pulmonary arterial und/or arterial hypertension and diseases of the heart valves. A preferred medical indication to be treated by the present invention is myocardial infarct extension.

“Symptoms” of a disease are implication of the disease noticeable by the tissue, organ or organism having such disease and include but are not limited to pain, weakness, tenderness, strain, stiffness, and spasm of the tissue, an organ or an individual. “Signs” or “signals” of a disease include but are not limited to the change or alteration such as the presence, absence, increase or elevation, decrease or decline, of specific indicators such as biomarkers or molecular markers, or the development, presence, or worsening of symptoms.

A disorder may be acquired or congenital. In this context, the term “acquired” means that the medical condition, i.e., the disorder, developed post-fetally. Such an acquired disorder in the context of the present invention may be a myocardial infarction. Congenital disorders involve defects to a developing fetus which may be the result of genetic abnormalities, errors of morphogenesis, or chromosomal abnormalities. Genetic diseases or disorders are all congenital, though they may not be expressed or recognized until later in life. Congenital disorders in the context of the present invention are, for example, Nemaline myopathy, Myotubular myopathy, or Centronuclear myopathy. Furthermore, in the context of the present invention, the cardiac disorder may be acute or chronic. For example, an acute cardiac disorder is acute heart failure, an acute skeletal muscle disorder is Rhabdomyolysis. A chronic cardiac muscle disease is, for example, chronic heart failure. The cardiac disorder may be due to the muscular malfunction which may be associated with defective calcium cycling and/or defective contractile performance in the muscle cells, preferably the cardiomyocytes.

As used herein, “treat”, “treating” or “treatment” of a disease or disorder means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in an individual that have previously had the disorder(s); and (e) limiting or preventing recurrence of symptoms in individuals that were previously symptomatic for the disorder(s).

As used herein, “prevent”, “preventing”, “prevention”, “prophylaxis” of a disease or disorder means preventing that such disease or disorder occurs in the patient.

As used herein, “administering” includes in vivo administration, as well as administration directly to tissue ex vivo, such as vein grafts.

An “effective amount” or a “therapeutically effective amount” is an amount of a therapeutic agent sufficient to achieve the intended purpose. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art.

The terms “pharmaceutical”, “medicament” and “drug” are used interchangeably herein referring to a substance and/or a combination of substances being used for the identification, prevention or treatment of a tissue status or disease.

“Pharmaceutically acceptable” means approved by a regulatory agency of the Federal or a state government or listed in the U.S. Pharmacopeia or other generally recognized pharmacopeia for use in animals, and more particularly in humans. The term “pharmaceutically acceptable salt” refers to a salt of the protein or peptide of the present invention. Suitable pharmaceutically acceptable salts include acid addition salts which may, for example, be formed by mixing a solution of the peptide of the present invention with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the peptide carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include, but are not limited to, acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like (see, for example, S. M. Berge et al., “Pharmaceutical Salts”, J. Pharm. Sci., 66, pp. 1-19 (1977)).

The term “active ingredient” refers to the substance in a pharmaceutic formulation that is biologically active, i.e. that provides pharmaceutical value. A pharmaceutical composition may comprise one or more active ingredients which may act in conjunction with or independently of each other. The active ingredient can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as but not limited to those derived from sodium, potassium, ammonium, calcium, ferric hydroxides, isopropylamine, triethylamine, 2-ethylamino ethanol, histidine, procaine, and the like.

Whether or not a fragment or variant of the S100A1 protein of the present invention is biologically active can, for example, be determined by any one of the tests described in the examples below. According to a preferred embodiment, a fragment or variant of the S100A1 protein is biologically active if the results obtained with such fragment or variant compared to the results obtained with the S100A1 protein of the present invention shown in at least one of the examples presented herein below achieve at least 50%, at least 60%, at least 70%, at least 80%, at least 90%, at least 95% or 100% of the effect reported for the S100A1 protein over the indicated controls.

The terms “preparation” and “composition” are intended to include the formulation of the active compound with encapsulating material as a carrier providing a capsule in which the active component with or without other carriers, is surrounded by a carrier, which is thus in association with it.

The term “carrier”, as used herein, refers to a pharmacologically inactive substance such as but not limited to a diluent, excipient, or vehicle with which the therapeutically active ingredient is administered. Such pharmaceutical carriers can be liquid or solid. Liquid carriers include but are not limited to sterile liquids, such as saline solutions in water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. A saline solution is a preferred carrier when the pharmaceutical composition is administered intravenously. Examples of suitable pharmaceutical carriers are described in “Remington's Pharmaceutical Sciences” by E. W. Martin.

Suitable pharmaceutical “excipients” include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The term “adjuvant” refers to agents that augment, stimulate, activate, potentiate, or modulate the immune response to the active ingredient of the composition at either the cellular or humoral level, e.g. immunologic adjuvants stimulate the response of the immune system to the actual antigen, but have no immunological effect themselves. Examples of such adjuvants include but are not limited to inorganic adjuvants (e.g. inorganic metal salts such as aluminium phosphate or aluminium hydroxide), organic adjuvants (e.g. saponins or squalene), oil-based adjuvants (e.g. Freund's complete adjuvant and Freund's incomplete adjuvant), cytokines (e.g. IL-1β, IL-2, IL-7, IL-12, IL-18, GM-CFS, and INF-γ) particulate adjuvants (e.g. immuno-stimulatory complexes (ISCOMS), liposomes, or biodegradable microspheres), virosomes, bacterial adjuvants (e.g. monophosphoryl lipid A, or muramyl peptides), synthetic adjuvants (e.g. non-ionic block copolymers, muramyl peptide analogues, or synthetic lipid A), or synthetic polynucleotides adjuvants (e.g polyarginine or polylysine).

### EMBODIMENTS

The S100A1 protein is a member of the S100 family of proteins expressed in cardiac muscle, skeletal muscle and brain. The present invention provides the S100A1 protein and biologically active fragments and variants thereof as a novel therapeutic factor that can prevent and treat the extension of the infarcted zone in the heart. Without wishing to be bound by any theory, S100A1 and its biologically active fragments and variants exert their positive effects by inhibiting the post-myocardial sterile inflammation and by lending molecular inotropic support to the damaged heart. The present inventors surprisingly found that the S100A1 protein and its biologically active fragments and variants attenuates both innate and adaptive immune system activity in the post-MI heart. The present inventors show inter alia that the therapeutic effect of the S100A1 protein and its biologically active fragments and variants is based on attenuating both the activity of innate and adaptive immune cell activities.

So far, the S100A1 protein had been suggested as a pure inotropic therapeutic in post-MI heart failure and cardiomyopathies, since it was shown that myocardial levels of S100A1 are decreased in heart failure and that S100A1 delivery to cardiomyocytes results in an increase of isometric contraction followed by an increase in the amount of calcium pumped into the sarcoplasmic reticulum (P. Most et al., “S100A1: a novel inotropic regulator of cardiac performance. Transition from molecular physiology to pathophysiological relevance”, Am J Physiol., 293, 2007; J. Ritterhoff & P. Most, “Targeting S100A1 in heart failure”, Gene Therapy, 19, 2012). The use of the S100A1 and its biologically active fragments and variants for treating and/or preventing myocardial infarct extension was hitherto unknown and resembles a further medical use defined by a new medical indication.

According to a first aspect, the present invention provides an S100A1 protein, a biologically active fragment or variant thereof, or a nucleic acid encoding said S100A1 protein or the biologically active fragment or variant thereof, for use in treating and/or preventing infarct extension in a patient in need thereof. The biologically active fragment or variant of the S100A1 protein has at least 80% sequence identity to SEQ ID NO:1. A respective fragment or variant may thus have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 1. For short fragments or deletion variants of SEQ ID NO:1, i.e. peptides having a length of between 9 and 15 amino acids of SEQ ID NO: 1, the fragment or deletion variant preferably has at least 88% sequence identity to SEQ ID NO:1.

Non-limiting examples of variants include but are not limited to S100A2, S100A3, S100A4 and other members of the S100 protein family. Respective variants comprising e.g. amino acid substitutions differ from SEQ ID NO: 1 preferably at the C-terminus of the protein, more preferably in one or more of the 20 most C-terminal amino acids. These variants may further be truncated in that they also comprise deletions if compared to SEQ ID NO: 1. In such a case, they are also referred to as deletion variants of SEQ ID NO: 1.

Fragments of SEQ ID NO: 1 according to the present invention differ from SEQ ID NO: 1 preferably at the N-terminus or internally and comprise one or more amino acid deletions. A particularly preferred fragment are peptides consisting of amino acids N65 to S94, V70 to S94, or Y75 to S94 of SEQ ID NO:1. Fragments may additionally comprise one or more substitution in their amino acid sequence compared to SEQ ID NO: 1. In such cases, the fragments are also referred to as deletion variants.

Preferably, the fragment or variant of SEQ ID NO: 1 maintains or exerts essentially the same biological function as the parent protein according to SEQ ID NO:1. Maintaining or exerting essentially the same biological function as the parent protein according to SEQ ID NO: 1 means, in accordance with the present invention, one or more of interacting with the RyR calcium release channel and sarcoplasmic/endoplasmic reticulum calcium ATPase (SERCA), increasing SERCA activity, increasing contractile function in cardiac muscle cells and/or skeletal muscle cells, exhibiting the inotropic effect of the full-length S100A1 protein (all described in Most P. et al., 2007, Am. J. Physiol. Regul. Integr. Comp. Physiol. 293:R568-577; Voelkers M. et al., 2007, Cell Calcium 41:135-143), and inhibiting post-myocardial sterile inflammation.

According to a preferred embodiment, however, the S100A1 protein is the naturally occurring human S100A1 protein having the amino acid sequence of SEQ ID NO: 1.

In a preferred embodiment, the protein further comprises one or more of the elements selected from the group consisting of a hydrophilic domain, a membrane penetration enhancing domain, one or more epitope-tag(s), and a peptide targeting domain, preferably a hydrophilic domain, a membrane penetration enhancing domain or a hydrophilic domain and a membrane penetration enhancing domain. These elements may be linked directly or indirectly to the N- or C-terminus of the S100A1 protein derived domain. Preferably, the element is linked to the N-terminus.

In one embodiment, the protein further comprises epitope-tag(s), and/or a peptide targeting domain. In another embodiment, the peptide further comprises one or more, e.g. one, two, three, or four of the elements selected from the group consisting of a hydrophilic domain, a membrane penetration enhancing domain, one or more epitope-tag(s), and a peptide targeting domain.

An epitope is a portion of a molecule to which an antibody binds. In the context of the present invention, an epitope is preferably a peptide-tag, for example, hemagglutinin-(HA-), FLAG-, myc-, or a poly-His-tag. Such an epitope tag may be used to locate the peptide of the present invention within a cell, for example, for determining whether the peptide penetrates, i.e., traverses, cell membranes and can be found inside an intact cell incubated with said peptide.

A peptide targeting domain in the context of the present invention may be any moiety that is suitable for targeting a peptide in vivo to a specific organ or specific cells. For example, a peptide targeting domain may be a peptide that specifically binds to a particular receptor which is specific for certain cells or a certain organ. Preferably, the presence of a peptide targeting domain within the peptide according to the present invention allows for specific targeting of cells or organs in a patient to which the peptide was administered systemically.

In embodiments of the present invention, the nucleic acid encoding the S100A1 protein or the fragment or variant thereof is comprised in a vector. Preferably, the vector is selected from the group consisting of plasmid vectors, cosmid vectors, phage vectors such as lambda phage, filamentous phage vectors, viral vectors, viral like particles, and bacterial spores. It is particularly preferred that the viral vector is selected from the group consisting of an adenoviral vector, adeno-associated viral (AAV) vector, alphaviral vector, herpes viral vector, measles viral vector, pox viral vector, vesicular stomatitis viral vector, retroviral vector and lentiviral vector. In preferred embodiments, the vector integrates into the genome, preferably into the genome of myocardial cells. In further preferred embodiments, the vector is an AAV selected from the group consisting of AAV5, AAV6 and AAV9. According to the most preferred embodiment, the nucleic acid encoding the S100A1 protein or the fragment or variant thereof is comprised in an AAV5 vector.

In preferred embodiments, the above described vector triggers the expression of the S100A1 protein or of the fragments or variants thereof in myocardial cells. In further preferred embodiments, the expression of the S100A1 protein is controlled by a heart tissue specific promoter. Thus, according to a preferred embodiment, the vector further comprises a heart tissue specific promoter. Preferably the heart tissue specific promoter is selected from the group consisting of but not limited to Cardiac Actin Enhancer/Elongation Factor 1 promoter, Cytomegolo-virus enhancer/Myosin light chain ventricle 2 promoter and Troponin.

In preferred embodiments, the vector is a viral vector which is preferably administered in a dosage of 1×10¹²-1×10¹⁴ vector genome copies, more preferably 3×10¹⁰ vgc, or around 3×10¹¹ vgc/kg body weight (vgc/kg BW) to around 3.3×10¹² vgc/kg BW, preferably around 3.3×10¹¹ vgc/kg BW.

In preferred embodiments of the present invention, the intracellular level of said S100A1 protein is raised in at least 30% of the cells of the heart tissue of said individual. The size and amount of treated subsections and, thus the amount of heart cells expressing the S100A1 protein will depend on the underlying disease condition. However, it is preferred that within a treated heart region the intracellular level of S100A1 is raised as described herein.

In embodiments of the present invention, the S100A1 protein or fragment or variant thereof, or the nucleic acid encoding said S100A1 protein or fragment or variant thereof, described herein, is administered through the oral, intravenous, intramucosal, intraarterial, intramuscular or intracoronal route. According to a particularly preferred embodiment, the protein, the nucleic acid, the vector or the pharmaceutical composition is administered retrograde into the coronary venous system of the patient.

According to one embodiment, the protein, the biologically active fragment or variant thereof, the vector or the pharmaceutical composition is administered between 1 day and four weeks after a myocardial infarction in said patient has become post-ischemic. Preferably, the protein, the biologically active fragment or variant thereof, the vector or the pharmaceutical composition is administered between one week and two weeks after a myocardial infarction in said patient has become post-ischemic.

In further embodiments of the present invention, the intracellular level of said S100A1 protein is raised for a period of at least 7 days, more preferably of at least 10 days, and even more preferably for a period of at least 14 days. Alternatively, the intracellular level of said S100A1 protein is raised for a period of at least one week, two weeks, three weeks, four weeks, five weeks, six weeks, seven weeks, eight weeks, nine weeks, ten weeks, eleven weeks, or twelve weeks or longer. This is preferably attained as a result of a single administration or repetitive administration. Single administration is preferred.

In embodiments of the present invention, the individual or patient has suffered or suffers from, or is at risk of developing a cardiac disorder. Preferably, the cardiac disorder myocardial infarction.

According a further aspect, the present invention provides a pharmaceutical composition comprising either (i) an S100A1 protein, a biologically active fragment thereof or a nucleic acid encoding said S100A1 protein, (ii) a vector comprising a nucleic acid encoding an S100A1 protein or a biologically active fragment or variant thereof having at least 80% sequence identity to SEQ ID NO: 1, or (iii) a pharmaceutically acceptable salt of (i) or (ii). The pharmaceutical composition is for use in treating and/or preventing infarct extension in a patient in need thereof.

In one embodiment, the pharmaceutical composition comprises a therapeutically effective amount of the S100A1 protein or its fragment or variant as described herein, the nucleic acid, or of the vector of the present invention, which are herein also referred to as the “active ingredient”. In one embodiment, the pharmaceutical composition comprises a therapeutically effective amount of the active ingredient, preferably in purified form, together with a suitable amount of a carrier and/or excipient so as to provide the form for proper administration to the patient. The formulation of the composition should suit the mode of administration. For intravenous administration, it is preferred that the carrier is an aqueous carrier. According to one embodiment, such an aqueous carrier is capable of imparting improved properties when combined with an antigen binding polypeptide of the invention, for example, improved solubility, efficacy, and/or improved immunotherapy.

According to one embodiment, the pharmaceutical composition may comprise a further therapeutic agent or pharmacologically active substance such as but not limited to adjuvants and/or additional active ingredients, in a pharmaceutically or physiologically acceptable formulation selected to be suitably administered according to the selected mode of administration.

According to one embodiment, the pharmaceutical compositions can take the form of solutions, suspensions, emulsion, tablets, pills, capsules, powders, sustained-release formulations and the like. For preparing pharmaceutical compositions of the present invention, pharmaceutically acceptable carriers can be either solid or liquid and are preferably liquid. Liquid form compositions include solutions, suspensions, and emulsions, for example, water, saline solutions, aqueous dextrose, glycerol solutions or water/propylene glycol solutions. For parenteral injections (e.g. intravenous, intra-arterial, intraosseous infusion, intramuscular, subcutaneous, intraperitoneal, intradermal, and intrathecal injections), liquid preparations can be formulated in solution in, e.g. aqueous polyethylene glycol solution. A saline solution is a preferred carrier when the pharmaceutical composition is to be administered intravenously.

According to one embodiment, the pharmaceutical composition is in a unit dosage form. In such form, the composition may be subdivided into unit doses containing appropriate quantities of the active component. The unit dosage form can be a packaged composition, the package containing discrete quantities of the composition, such as packaged tablets, capsules, and powders in vials or ampoules. Also, the unit dosage form can be a capsule, an injection vial, a tablet, a cachet, or a lozenge itself, or it can be the appropriate number of any of these in packaged form. The pharmaceutical composition, if desired, can also contain minor amounts of wetting or emulsifying agents, or pH buffering agents.

The form of the pharmaceutical compositions, the route of administration, the dosage and the regimen naturally depend upon the condition to be treated, the severity of the illness, the age, weight, and gender of the patient, the desired duration of the treatment etc. This pharmaceutical composition may be in any suitable form depending on the desired method of administering it to a patient. A preferred mode of administration is retrograde administration into the coronary venous system of the patient

According to one embodiment, the pharmaceutical composition comprises vehicles, which are pharmaceutically acceptable for a formulation capable to be injected into a patient. These may be in particular isotonic, sterile, saline solutions (monosodium or disodium phosphate, sodium, potassium, calcium or magnesium chloride and the like or mixtures of such salts) or dry, especially freeze-dried compositions which upon addition, depending on the case, of sterilized water or physiological saline, permit the constitution of injectable solutions.

To prepare pharmaceutical compositions, an effective amount of the active ingredient may be dissolved or dispersed in a pharmaceutically acceptable carrier or in an aqueous medium.

The pharmaceutical forms suitable for injectable use include sterile aqueous solutions or dispersions; formulations including sesame oil, peanut oil or aqueous propylene glycol; and sterile powders for the preparation of sterile injectable solutions or dispersions. In all cases, the form must be sterile and must be fluid to the extent that easy syringeability exists. It must be stable under the conditions of manufacture and storage and must be preserved against the contaminating action of microorganisms, such as bacteria and fungi. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

According to one embodiment, the S100A1 protein or its fragment or variant described herein, the nucleic acid, or the vector of the present invention can be formulated into a pharmaceutical composition in a neutral or salt form using pharmaceutically acceptable salts.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

According to a preferred embodiment, the pharmaceutical composition is administered through the oral, intravenous, intramucosal, intraarterial, intramuscular or intracoronal route. Intravenous administration is preferred. In cases of intravenous administrations, it is particularly preferred that the protein, the nucleic acid, the vector or the pharmaceutical composition is administered retrograde into the coronary venous system of the patient.

In preferred embodiments, administration of the protein, the biologically active fragment thereof, the nucleic acid, the vector or the pharmaceutical composition of the present invention results in an increase in the concentration of the S100A1 protein in the myocardium of said individual about 50-fold compared to the concentration of S100A1 in the myocardium of a healthy individual. Thus, preferably, the concentration of the S100A1 protein within the myocardium is increased by 2-fold, 3-fold, 4-fold, 5-fold, 6-fold, 7-fold, 8-fold, 9-fold, 10-fold, 11-fold, 12-fold, 13-fold, 14-fold, 15-fold, 20-fold, 25-fold, 30-fold, 35-fold, 40-fold, 45-fold, or 50-fold relative to the concentration of S100A1 in the myocardium of a healthy individual.

According to a further aspect, the present invention relates to a method for treating and/or preventing infarct extension in a patient in need thereof, the method comprising administering to the patient either (i) an S100A1 protein, a biologically active fragment or variant thereof or a nucleic acid encoding said S100A1 protein, the fragment or variant thereof, (ii) a vector comprising a nucleic acid encoding an S100A1 protein or a biologically active fragment or variant thereof, or (iii) a pharmaceutically acceptable salt of (i) or (ii). The biologically active fragment or variant of the S100A1 protein has at least 80% sequence identity to SEQ ID NO:1. A respective fragment or variant may thus have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO:1. Non-limiting examples of such fragments or variants include but are not limited to S100A2, S100A3, S100A4 and other members of the S100 protein family. Respective fragments and variants differ from SEQ ID NO:1 preferably at the C-terminus of the protein, more preferably in one or more of the 20 most C-terminal amino acids. Preferably, the fragment or variant of SEQ ID NO: 1 further maintains or exerts essentially the same biological function as the parent protein according to SEQ ID NO:1.

According to a preferred embodiment, however, the S100A1 protein is the naturally occurring human S100A1 protein having the amino acid sequence of SEQ ID NO:1.

In a preferred embodiment, the protein further comprises one or more of the elements selected from the group consisting of a hydrophilic domain, a membrane penetration enhancing domain, one or more epitope-tag(s), and a peptide targeting domain, preferably a hydrophilic domain, a membrane penetration enhancing domain or a hydrophilic domain and a membrane penetration enhancing domain. These elements may be linked directly or indirectly to the N- or C-terminus of the S100A1 protein derived domain. Preferably, the element is linked to the N-terminus.

In one embodiment, the protein further comprises epitope-tag(s), and/or a peptide targeting domain. In another embodiment, the peptide further comprises one or more, e.g. one, two, three, or four of the elements selected from the group consisting of a hydrophilic domain, a membrane penetration enhancing domain, one or more epitope-tag(s), and a peptide targeting domain.

An epitope is a portion of a molecule to which an antibody binds. In the context of the present invention, an epitope is preferably a peptide-tag, for example, hemagglutinin-(HA-), FLAG-, myc-, or a poly-His-tag. Such an epitope tag may be used to locate the peptide of the present invention within a cell, for example, for determining whether the peptide penetrates, i.e., traverses, cell membranes and can be found inside an intact cell incubated with said peptide.

A peptide targeting domain in the context of the present invention may be any moiety that is suitable for targeting a peptide in vivo to a specific organ or specific cells. For example, a peptide targeting domain may be a peptide that specifically binds to a particular receptor which is specific for certain cells or a certain organ. Preferably, the presence of a peptide targeting domain within the peptide according to the present invention allows for specific targeting of cells or organs in a patient to which the peptide was administered systemically.

In embodiments of the present invention, the nucleic acid encoding the S100A1 protein or the fragment or variant thereof is comprised in a vector. Preferably, the vector is selected from the group consisting of plasmid vectors, cosmid vectors, phage vectors such as lambda phage, filamentous phage vectors, viral vectors, viral like particles, and bacterial spores. It is particularly preferred that the viral vector is selected from the group consisting of an adenoviral vector, adeno-associated viral (AAV) vector, alphaviral vector, herpes viral vector, measles viral vector, pox viral vector, vesicular stomatitis viral vector, retroviral vector and lentiviral vector. In preferred embodiments, the vector integrates into the genome, preferably into the genome of myocardial cells. In further preferred embodiments, the vector is an AAV selected from the group consisting of AAV5, AAV6 and AAV9. According to the most preferred embodiment, the nucleic acid encoding the S100A1 protein or the fragment or variant thereof is comprised in an AAV5 vector.

In preferred embodiments, the above described vector triggers the expression of the S100A1 protein or of the fragments or variants thereof in myocardial cells. In further preferred embodiments, the expression of the S100A1 protein is controlled by a heart tissue specific promoter. Thus, according to a preferred embodiment, the vector further comprises a heart tissue specific promoter. Preferably the heart tissue specific promoter is selected from the group consisting of but not limited to Cardiac Actin Enhancer/Elongation Factor 1 promoter, Cytomegolo-virus enhancer/Myosin light chain ventricle 2 promoter and Troponin.

In preferred embodiments, the vector is a viral vector which is preferably administered in a dosage of between 1×10¹¹ and 1×10¹⁴ vector genome copies (vgc), preferably between 2×10¹¹ and 1×10¹³ vgc, or around 3.3×10¹⁰ vgc/kg body weight (vgc/kg BW) to around 3.3×10¹² vgc/kg BW, preferably around 3.3×10¹¹ vgc/kg BW.

In preferred embodiments of the present invention, the intracellular level of said S100A1 protein is raised in at least 30% of the cells of the heart tissue of said individual. The size and amount of treated subsections and, thus the amount of heart cells expressing the S100A1 protein will depend on the underlying disease condition. However, it is preferred that within a treated heart region the intracellular level of S100A1 is raised as described herein.

In embodiments of the present invention, the S100A1 protein or fragment or variant thereof, or the nucleic acid encoding said S100A1 protein or fragment or variant thereof, described herein, is administered through the oral, intravenous, intramucosal, intraarterial, intramuscular or intracoronal route. According to a particularly preferred embodiment, the protein, the nucleic acid, the vector or the pharmaceutical composition is administered retrograde into the coronary venous system of the patient.

According to one embodiment, the protein, the biologically active fragment or variant thereof, the vector or the pharmaceutical composition is administered between 1 day and four weeks after a myocardial infarction in said patient has become post-ischemic. Preferably, the protein, the biologically active fragment or variant thereof, the vector or the pharmaceutical composition is administered between one week and two weeks after a myocardial infarction in said patient has become post-ischemic.

In further embodiments of the present invention, the intracellular level of said S100A1 protein is raised for a period of at least 7 days, more preferably of at least 10 days, and even more preferably for a period of at least 14 days. Alternatively, the intracellular level of said S100A1 protein is raised for a period of at least one week, two weeks, three weeks, four weeks, five weeks, six weeks, seven weeks, eight weeks, nine weeks, ten weeks, eleven weeks, or twelve weeks or longer. This is preferably attained as a result of a single administration or repetitive administration. Single administration is preferred.

In embodiments of the present invention, the individual or patient has suffered or suffers from, or is at risk of developing a cardiac disorder. Preferably, the cardiac disorder myocardial infarction.

In one embodiment, the pharmaceutical composition comprises a therapeutically effective amount of the S100A1 protein or its fragment or variant as described herein, the nucleic acid, or of the vector of the present invention, which are herein also referred to as the “active ingredient”. In one embodiment, the pharmaceutical composition comprises a therapeutically effective amount of the active ingredient, preferably in purified form, together with a suitable amount of a carrier and/or excipient so as to provide the form for proper administration to the patient. The formulation of the composition should suit the mode of administration. For intravenous administration, it is preferred that the carrier is an aqueous carrier. According to one embodiment, such an aqueous carrier is capable of imparting improved properties when combined with an antigen binding polypeptide of the invention, for example, improved solubility, efficacy, and/or improved immunotherapy.

According to one embodiment, the pharmaceutical composition may comprise a further therapeutic agent or pharmacologically active substance such as but not limited to adjuvants and/or additional active ingredients, in a pharmaceutically or physiologically acceptable formulation selected to be suitably administered according to the selected mode of administration.

According to one embodiment, the pharmaceutical composition can take the form of solutions, suspensions, emulsion, tablets, pills, capsules, powders, sustained-release formulations and the like. For preparing pharmaceutical compositions of the present invention, pharmaceutically acceptable carriers can be either solid or liquid and are preferably liquid. Liquid form compositions include solutions, suspensions, and emulsions, for example, water, saline solutions, aqueous dextrose, glycerol solutions or water/propylene glycol solutions. For parenteral injections (e.g. intravenous, intra-arterial, intraosseous infusion, intramuscular, subcutaneous, intraperitoneal, intradermal, and intrathecal injections), liquid preparations can be formulated in solution in, e.g. aqueous polyethylene glycol solution. A saline solution is a preferred carrier when the pharmaceutical composition is to be administered intravenously.

According to one embodiment, the pharmaceutical composition is in a unit dosage form. In such form, the composition may be subdivided into unit doses containing appropriate quantities of the active component. The unit dosage form can be a packaged composition, the package containing discrete quantities of the composition, such as packaged tablets, capsules, and powders in vials or ampoules. Also, the unit dosage form can be a capsule, an injection vial, a tablet, a cachet, or a lozenge itself, or it can be the appropriate number of any of these in packaged form. The pharmaceutical composition, if desired, can also contain minor amounts of wetting or emulsifying agents, or pH buffering agents.

The form of the pharmaceutical compositions, the route of administration, the dosage and the regimen naturally depend upon the condition to be treated, the severity of the illness, the age, weight, and gender of the patient, the desired duration of the treatment etc. This pharmaceutical composition may be in any suitable form depending on the desired method of administering it to a patient. A preferred mode of administration is retrograde administration into the coronary venous system of the patient

According to one embodiment, the pharmaceutical composition comprises vehicles, which are pharmaceutically acceptable for a formulation capable to be injected into a patient. These may be in particular isotonic, sterile, saline solutions (monosodium or disodium phosphate, sodium, potassium, calcium or magnesium chloride and the like or mixtures of such salts) or dry, especially freeze-dried compositions which upon addition, depending on the case, of sterilized water or physiological saline, permit the constitution of injectable solutions.

To prepare pharmaceutical compositions, an effective amount of the active ingredient may be dissolved or dispersed in a pharmaceutically acceptable carrier or in an aqueous medium.

The pharmaceutical forms suitable for injectable use include sterile aqueous solutions or dispersions; formulations including sesame oil, peanut oil or aqueous propylene glycol; and sterile powders for the preparation of sterile injectable solutions or dispersions. In all cases, the form must be sterile and must be fluid to the extent that easy syringeability exists. It must be stable under the conditions of manufacture and storage and must be preserved against the contaminating action of microorganisms, such as bacteria and fungi. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

According to one embodiment, the S100A1 protein or its fragment or variant described herein, the nucleic acid, or the vector of the present invention can be formulated into a pharmaceutical composition in a neutral or salt form using pharmaceutically acceptable salts.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

According to a preferred embodiment, the pharmaceutical composition is administered through the oral, intravenous, intramucosal, intraarterial, intramuscular or intracoronal route. Intravenous administration is preferred. In cases of intravenous administrations, it is particularly preferred that the protein, the nucleic acid, the vector or the pharmaceutical composition is administered retrograde into the coronary venous system of the patient.

In preferred embodiments, administration of the protein, the biologically active fragment thereof, the nucleic acid, the vector or the pharmaceutical composition of the present invention results in an increase in the concentration of the S100A1 protein in the myocardium of said individual about 50-fold compared to the concentration of S100A1 in the myocardium of a healthy individual. Thus, preferably, the concentration of the S100A1 protein within the myocardium is increased by 2-fold, 3-fold, 4-fold, 5-fold, 6-fold, 7-fold, 8-fold, 9-fold, 10-fold, 11-fold, 12-fold, 13-fold, 14-fold, 15-fold, 20-fold, 25-fold, 30-fold, 35-fold, 40-fold, 45-fold, or 50-fold relative to the concentration of S100A1 in the myocardium of a healthy individual.

The present invention further relates to the following items:

Item 1: An S100A1 protein, a biologically active fragment or variant thereof or a nucleic acid encoding said S100A1 protein or the biologically active fragment or variant thereof, for use in treating and/or preventing infarct extension in a patient in need thereof, wherein the fragment or variant has at least 80% sequence identity to SEQ ID NO:1.

Item 2.: A vector comprising a nucleic acid encoding an S100A1 protein or a biologically active fragment or variant thereof having at least 80% sequence identity to SEQ ID NO:1, for use in treating and/or preventing infarct extension in a patient in need thereof.

Item 3: A pharmaceutical composition comprising
(i) an S100A1 protein, a biologically active fragment or variant thereof having at least 80% sequence identity to SEQ ID NO:1 or a nucleic acid encoding said S100A1 protein, fragment or variant;
(ii) a vector comprising a nucleic acid encoding an S100A1 protein or a biologically active fragment or variant thereof having at least 80% sequence identity to SEQ ID NO:1; or
(iii) a pharmaceutically acceptable salt of (i) or (ii),
for use in treating and/or preventing infarct extension in a patient in need thereof, optionally wherein the pharmaceutical composition further comprises a pharmaceutically acceptable excipient, carrier, and/or diluent.

Item 4.: The S100A1 protein, the biologically active fragment or variant thereof, the nucleic acid, the vector or the pharmaceutical composition for use according to one of items 1 to 3, wherein the S100A1 protein has the sequence as set forth in SEQ ID NO: 1.

Item 5: The vector or the pharmaceutical composition for use according to item 2, 3 or 4, wherein the vector is selected from the group consisting of plasmid vectors, cosmid vectors, phage vectors such as lambda phage, filamentous phage vectors, viral vectors, viral like particles, and bacterial spores.

Item 6: The vector or the pharmaceutical composition for use according to item 5, wherein the viral vector is selected from the group consisting of an adenoviral vector, adeno-associated viral (AAV) vector, alphaviral vector, herpes viral vector, measles viral vector, pox viral vector, vesicular stomatitis viral vector, retroviral vector and lentiviral vector.

Item 7.: The vector or the pharmaceutical composition for use according to item 6, wherein the adeno-associated viral (AAV) vector is selected from the group consisting of AAV5, AAV6, and AAV9, preferably AAV5.

Item 8.: The vector or the pharmaceutical composition for use according to any one of items 2 to 7, wherein the expression of the S100A1 protein is controlled by a heart tissue specific promoter.

Item 9.: The vector or the pharmaceutical composition for use according to item 8, wherein the heart-tissue specific promoter is selected from the group consisting of Cardiac Actin Enhancer/Elongation Factor 1 promoter Cytomegalo-virus enhancer/Myosin light chain ventricle 2 promoter and Troponin.

Item 10: The S100A1 protein, the biologically active fragment or variant thereof, the nucleic acid, the vector or the pharmaceutical composition for use according to any one of items 1 to 9, wherein the protein, the biologically active fragment thereof, the nucleic acid, the vector or the pharmaceutical composition is administered via the oral, intravenous, intramucosal, intraarterial, intramuscular or intracoronal route.

Item 11: The S100A1 protein, the biologically active fragment or variant thereof, the nucleic acid, the vector or the pharmaceutical composition for use according to item 10, wherein the protein, the biologically active fragment or variant thereof, the nucleic acid, the vector or the pharmaceutical composition is administered via the intravenous route, preferably wherein the protein, the nucleic acid, the vector or the pharmaceutical composition is administered retrograde into the coronary venous system of the patient.

Item 12: The S100A1 protein, the biologically active fragment or variant thereof, the nucleic acid, the vector or the pharmaceutical composition for use according to any one of items 1 to 11, wherein the protein, the biologically active fragment or variant thereof, the vector or the pharmaceutical composition is administered between 1 day and four weeks after a myocardial infarction in said patient has become post-ischemic.

Item 13.: The S100A1 protein, the biologically active fragment or variant thereof, the nucleic acid, the vector or the pharmaceutical composition for use according to any one of items 1 to 12, wherein the intracellular level of the S100A1 protein is raised for a period of at least 7 days.

Item 14: The vector or the pharmaceutical composition for use according to any one of items 2 to 13, wherein the vector is administered in a single dose of 1×10¹³ vector genomic copies (vgc).

Item 15: Method for treating and/or preventing infarct extension in a patient in need thereof, the method comprising administering to the patient:
(i) an S100A1 protein, a biologically active fragment or variant thereof, or a nucleic acid encoding said S100A1 protein or said fragment or variant thereof,
(ii) a vector comprising a nucleic acid encoding an S100A1 protein or a biologically active fragment thereof, or
(iii) a pharmaceutically acceptable salt of (i) or (ii), wherein the fragment or variant has at least 80% sequence identity to SEQ ID NO:1.

Item 16: The method according to item 15, wherein the S100A1 protein has the sequence as set forth in SEQ ID NO:1.

Item 17: The method according to item 15, wherein the vector is selected from the group consisting of plasmid vectors, cosmid vectors, phage vectors such as lambda phage, filamentous phage vectors, viral vectors, viral like particles, and bacterial spores.

Item 18: The method according to item 17, wherein the viral vector is selected from the group consisting of an adenoviral vector, adeno-associated viral (AAV) vector, alphaviral vector, herpes viral vector, measles viral vector, pox viral vector, vesicular stomatitis viral vector, retroviral vector and lentiviral vector.

Item 19: The method according to item 18, wherein, wherein the adeno-associated viral (AAV) vector is selected from the group consisting of AAV5, AAV6, and AAV9.

Item 20: The method according to item 15, wherein the expression of the S100A1 protein is controlled by a heart tissue specific promoter.

Item 21: The method according to claim 20, wherein the heart-tissue specific promoter is selected from the group consisting of Cardiac Actin Enhancer/Elongation Factor 1 promoter Cytomegalo-virus enhancer/Myosin light chain ventricle 2 promoter and Troponin.

Item 22: The method according to claim 15, wherein the protein, the biologically active fragment thereof, the nucleic acid, the vector or the pharmaceutical composition is administered via the oral, intravenous, intramucosal, intraarterial, intramuscular or intracoronal route.

Item 23: The method according to item 22, wherein the protein, the biologically active fragment thereof, the nucleic acid, the vector or the pharmaceutical composition is administered via the intravenous route, preferably wherein the protein, the nucleic acid, the vector or the pharmaceutical composition is administered retrograde into the coronary venous system of the patient.

Item 24: The method according to item 15, wherein the protein, the biologically active fragment thereof, the vector or the pharmaceutical composition is administered between 1 day and four weeks after a myocardial infarction in said patient has become post-ischemic.

Item 25: The method according to item 15, wherein the intracellular level of the S100A1 protein is raised for a period of at least 7 days.

Item 26: The method according to item 15, wherein the vector is administered in a single dose of 1×10¹³ vector genomic copies (vgc).

### EXAMPLES

The Examples are designed to further illustrate the present invention and to serve a better understanding. They are not to be construed as limiting the scope of the invention in any way.

### Example 1

Myocardial Distribution of rAAV5, rAAV6 and rAAV9 after Cardiac-Targeted Retrograde Intravenous Administration in Pigs

It was investigated whether cardiac-targeted catheter-based retrograde intravenous delivery (CRID) of rAAV5—as a clinically applicable route of administration (ROA)-might result in a suitable in vivo cardiac transduction pattern of the porcine heart. To this end, a rAAV5 vector carrying a luciferase (luc) reporter gene was employed, which expression was controlled by a previously published cardiac-specific MLC-2v-driven promoter (P. Raake et al., “AAV6-BARKct cardiac gene therapy ameliorates cardiac function and normalizes the catecholaminergic axis in a clinically relevant large animal heart failure model”, Eur Heart J., 34, 2013). Biodistribution of rAAV5-luc was systematically assessed 4 weeks after CRID in healthy pigs by luminometric assessment of luc reporter gene activity in porcine heart and further organ samples. These results were benchmarked against CRID of rAAV6-luc and rAAV9-luc given their previously demonstrated ability to transduce diseased porcine hearts e.g., with the hs100a1 gene (P. Most et al., “Cardiac AAV9-S100A1 gene therapy rescues post-ischemic heart failure in a preclinical large animal model”, Science Translational Medicine, 3, 2011; C. Weber, “Therapeutic safety of high myocardial expression levels of the molecular inotrope S100A1 in a preclinical heart failure model”, 21, 2014). Anesthetized and ventilated pigs received 1.0×10¹³ vgc of either rAAV5-luc, rAAV6-luc and rAAV9-luc. As part of a standardized operational procedure (SOP) with reproducible quality measures for CRID, appropriate positioning of the retroinfusion catheter in the cardiac anterior interventricular vein (AIV) proximal of the outflow of the coronary sinus was recorded by digital fluoroscopic images. Then, contrast agent was retrogradely administered over the blocked venous catheter to rule out an immediate leakage into the systemic circulation and avoidance of a selective transfer into smaller venous branches of the AIV during rAAV infusion. Concomitantly, the adequate position of the intracoronary balloon catheter was ascertained to avert an immediate antegrade flushing of the myocardial capillary system during the retrograde rAAV installation. For each animal, digital fluoroscopic images each part of the CRID SOP were recorded and stored.

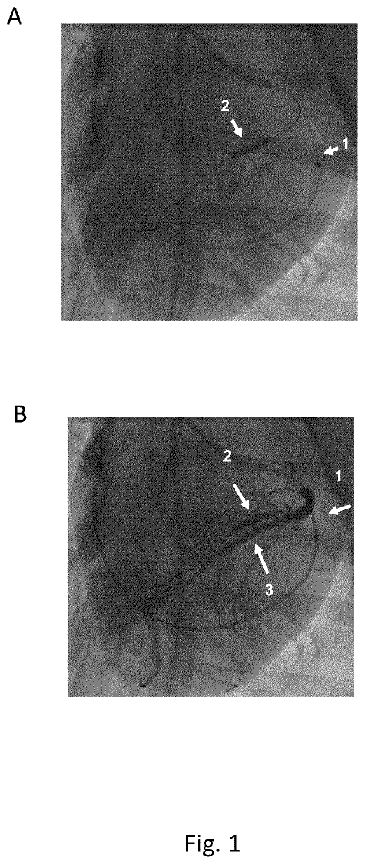

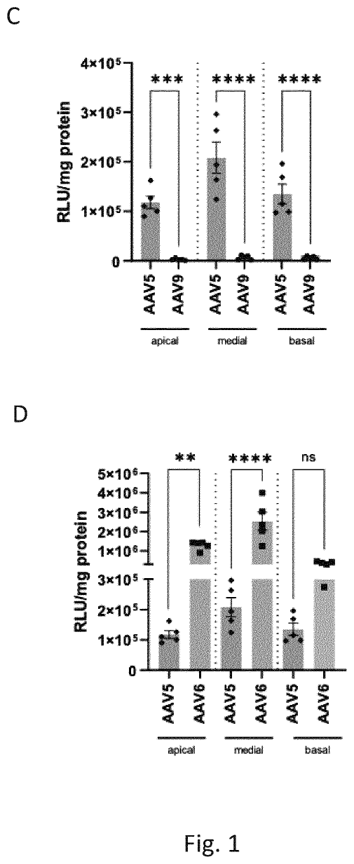

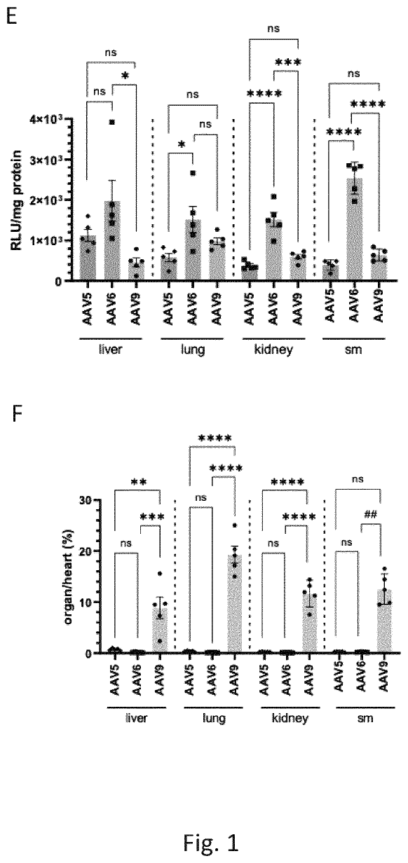

FIGS. 1A and B illustrate representative fluoroscopic images documenting the aforementioned steps prior to CRID. FIGS. 1C and D depict the key findings for the cardiac and non-cardiac biodistribution of rAAV5, rAAV6 and rAAV9 by the systematic luminometric assessment of tissue luc activity. Due to the average myocardial luc activity levels, rAAV5 exerted an approximately 30-fold higher reporter gene activity than rAAV9, which is widely used for preclinical cardiac GTMP development (rAAV5 167.179=16.083 RLU/mg protein vs. rAAV9 5.084±835 RLU/mg protein; data are given as mean±SEM, P<0.01 rAAV5 vs. rAAV9). FIGS. 1E and F show that the CRID SOP is associated with significantly lower luc activity of each tested rAAV serotype in liver, lung, kidney and skeletal muscle. These results indicate that inter alia rAAV5 is a preferred vector for cardiac-targeted hs100a1 gene therapy via CRID as a clinically applicable ROA in a relevant cardiac disease model that closely approximates human pathophysiology.

### Example 2

Impact of rAAV5-Hs100a1 on Cardiac Performance and Damage in the Cardiac Injury Pig Model Assessed by CMR

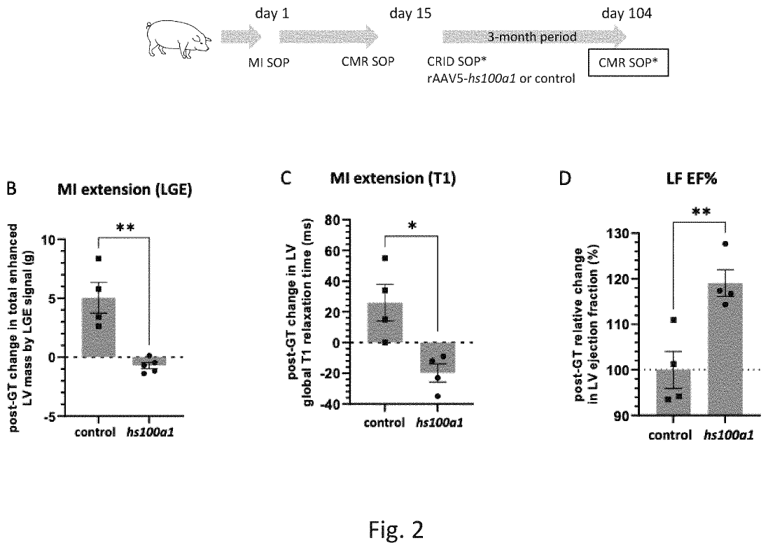

MI size was again assessed by LGE and additionally by global native Tl-weighing 2 weeks after experimental MI and only pigs with an MI size >14% of the LV were alternately assigned to the rAAV5-luc control and rAAV5-hs100a1 intervention. Each animal received a single dosage of 1.0×10¹³ vgc of either vector to benchmark the study against the previously conducted rAAV9-hs100a1 and rAAV6-hs100a1 studies. The CRID SOP for rAAV5 was performed as described in these studies (P. Most et al., “Cardiac AAV9-S100A1 gene therapy rescues post-ischemic heart failure in a preclinical large animal model”, Science Translational Medicine, 3, 2011; C. Weber, “Therapeutic safety of high myocardial expression levels of the molecular inotrope S100A1 in a preclinical heart failure model”, 21, 2014) with the herein established quality measures and experimenters blinded to the intervention type. Design of the intervention study is shown in FIG. 2A. 3-months after rAAV5-based gene therapy, the CMR SOP was reapplied blinded to the observer to determine cardiac performance and MI size changes within and among both groups. LV-EF and MI mass served as co-primary endpoints. Obtained data was concurrently analyzed and enrollment of animals stopped once a significant difference for one of the endpoints was reached against control.

FIG. 2A shows the general outline of the study, and FIG. 2B shows that rAAV5-hs100a1 treatment 2 weeks post-MI prevented the extension of total LV MI mass that had occurred in the control group until the end of the 12-week follow-up period, as assessed by the LGE signal and the Tl relaxation time. Concurrent CMR-based analysis of LV-EF changes unveiled a significant increase in LV-EF in the rAAV5-hs100a1 treated arm over rAAV5-luc (FIG. 2D). Significance for both co-primary endpoints was already reached with animal numbers ≤5 per group corroborating the implementation of standardized MI size cut-off and CMR protocols. It was concluded from this outcome that rAAV5 is a suitable rAAV serotype to sufficiently deliver the hs100a1 gene to human-sized diseased hearts and subsequently entail long-term improvement of cardiac performance. The yet unreported protective action of hs100a1 against MI extension actually sheds new light on the therapeutic potential of hs100a1 inasmuch the anti-inflammatory action of S100A1 improves post-MI overall cardiac contractile performance—as documented by the positive LF-EF change- and thereby halts MI extension.

### Example 3

S100A1 Expression Analyses, Exploratory Safety and Toxicity Assessment after rAAV5-Hs100a1 Cardiac-Targeted Delivery

First, cardiac expression of the therapeutic hs100a1 transgene was determined via semi-quantitative PCR to distinguish between human and porcine S100A1 gene expression and to confirm effective myocardial gene delivery by cardiac-targeted CRID. Then, the rAAV5 off-target organ transduction noted in FIG. 1E and F (rAAV5 biodistribution) prompted an extra-cardiac hs100a1 expression analysis.

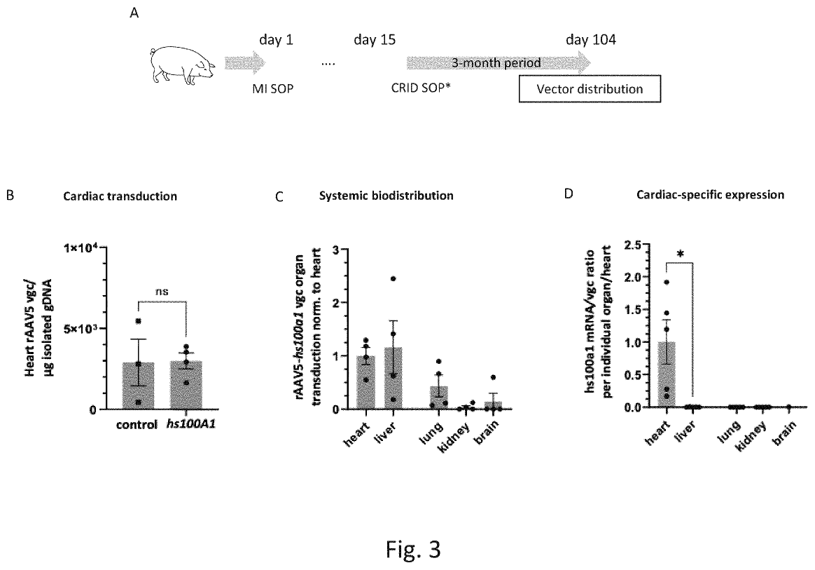

The experimental set-up is shown in FIG. 3A. A systemic assessment of the vector distribution (black encircled box) was conducted from biosamples of each animal 3-months after rAAV5-hs100a1 gene therapy. FIG. 3B confirms similar transgene expression levels in control and rAAV5-s100a1 treated hearts after the CRID SOP. Detection of hs100a1 mRNA e.g., in liver, lung, kidney or CNS suggests that rAAV5 that is not absorbed by the heart during the CRID SOP eventually reaches the systemic circulation and can result in extra-cardiac transduction (FIG. 3C). FIG. 3D confirms that hs100a1 mRNA could not be detected in other organs than the heart, which supports the notion that the cardiac-biased promoter contained hs100a1 expression to the heart despite some rAAV5 off-target transduction. Regular inspections of animal behavior, body temperature as well as food intake and excrement did not yield any abnormalities within the 12-week post-treatment observation period. It was concluded from these results that rAAV5 together with catheter-based CRID is suitable to deliver sufficient hs100a1 gene copies to the diseased pig heart to exert the demonstrated therapeutic effect on LV-EF % and MI extension in the clinically relevant dosage used.

### Example 4

Weighted Gene Co-Expression Network Analysis Indicates Active Cardioprotective and Attenuation of Inflammatory Gene Programs after rAAV5-Hs100a1 Based Gene Therapy

Aiming at further molecular clues for the superior study outcome, an unbiased network-based analytical approach was employed to account for the complex biological processes underlying the prevention of LV-MI extension and LV-EF improvement by rAAV5-hs100a1 treatment. To this end, bulk RNA-sequencing was carried out 12 weeks after gene transfer from LV myocardial tissue samples obtained from each enrolled animal. Then, a weighted gene correlation network analysis (WGCNA) was performed by applying a recently published WGCNA R software package (P. Langfelder & S. Horvath, “WGCNA: an R package for weighted correlation network analysis”, BMC Bioinformatics, 9, 2008) to capture clusters of highly co-expressed genes ensembles further referred to as modules. The module-trait relationships (MTRs) were subsequently calculated by pairwise correlation of each module's eigengene (ME) with the post-MI directed changes of LV-MI and LV-EF, respectively. Only modules with a significant and strong correlation (>0.8 and <−0.8) with LV-MI extension and/or LV-EF were further processed by in-silico pathway databases, such as Reactome, GEO and KEGG, to infer molecular pathways with potential mechanistic relevance for rAAV5-hs100a1 mediated therapeutic effects.

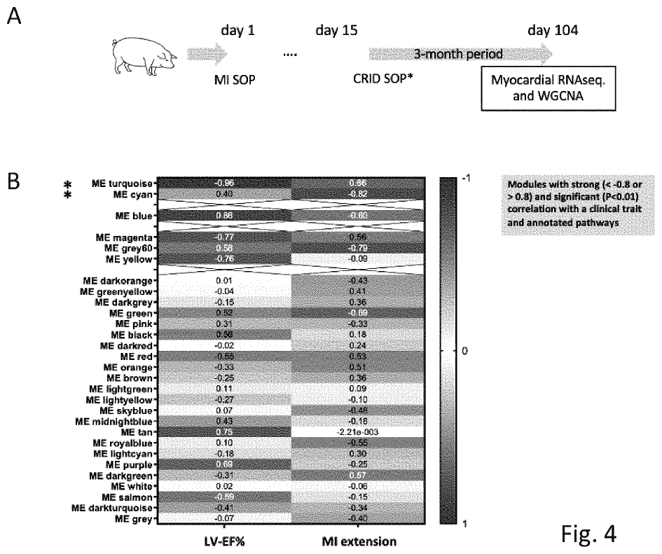

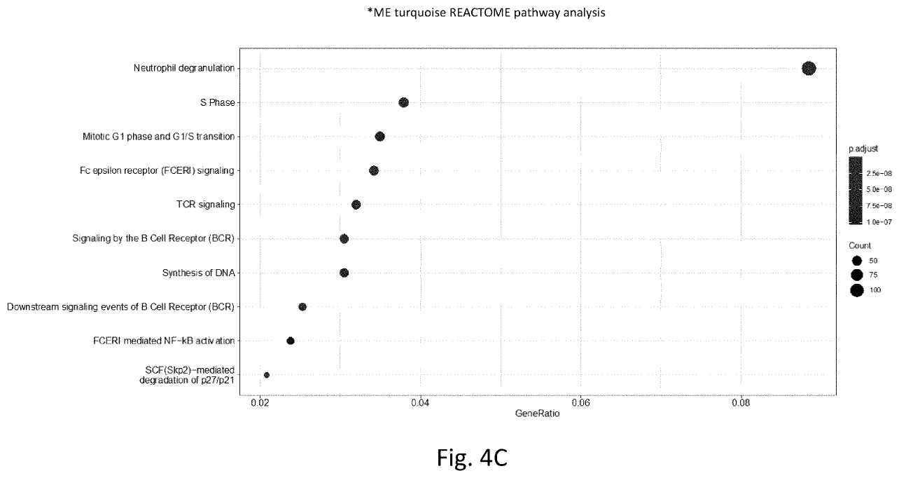

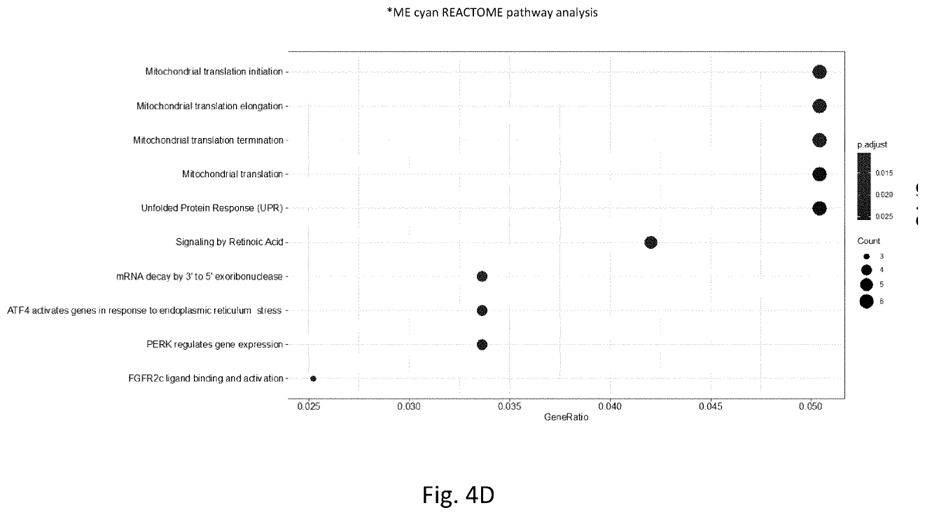

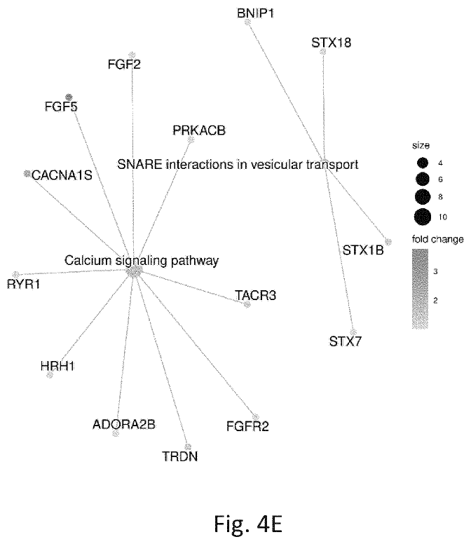

FIG. 4A shows the experimental set-up. Bulk myocardial tissue RNAseq and subsequent a WGCNA (black encircled box) was conducted 3-months after rAAV5-hs100a1 gene therapy. FIG. 4B, illustrating the MTR matrix, depicts the significant and strong correlation of the turquoise and cyan MEs with LV-EF and LV-MI extension, respectively. More specifically, ME turquoise exhibits a negative correlation with LV-EF changes in our model. Subsequent in silico pathway analysis, employing the Reactome, KEGG and GO term pathway databases, yielded a significant overrepresentation of the inflammatory and immunological signaling pathways “neutrophil degranulation”, “T cell receptor signaling”, “signaling by the B cell receptor”, “downstream signaling events of B cell receptor”, “Fc epsilon receptor signaling” and “Fc epsilon receptor signaling mediated NF-κB activation” in the turquoise ME (FIG. 4C). As such, the in-silico examination infers a lower activity of neutrophil as well as T- and B-cell related gene networks in rAAV5-hs100a1 treated porcine hearts where post-MI LV-EF was significantly enhanced.

Moreover, the MTR matrix disclosed a strong negative correlation of ME cyan with the LV-MI CMR surrogate Tl changes. Subsequent functional annotation, employing the Reactome, KEGG and GO term pathway databases, unveiled a significant overrepresentation of the pathways “calcium signaling”, “mitochondrial translation initiation, elongation and termination” as well as “ATF4 activates genes in response to endoplasmic reticulum stress”, “PERK regulates gene expression” and “unfolded protein response” as well as “signaling by retinoic acid” and “fibroblast growth factor receptor 2 ligand binding and activation” pathways in the cyan ME (FIGS. 4D and 4E). The system's transcriptome data analysis therefore infers a beneficial cooperation of cardiac calcium cycling, maintenance of mitochondrial energetic balance and stress responses with cardioprotective pathways in rAAV5-hs100a1 treated myocardium where post-MI LV-MI extension was completely blunted.

### Example 5

rAAV5-Hs100a1 Treatment of LV Infarcted Mice Recapitulates Attenuated Innate and Adaptive Immune System Activity in Myocardium

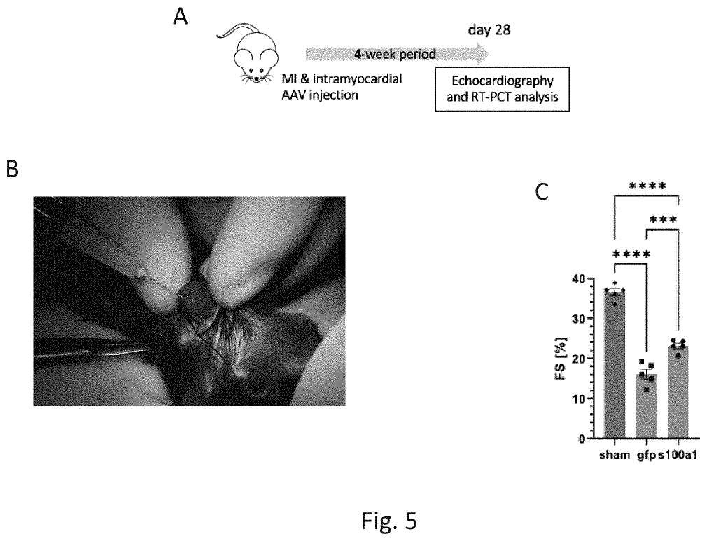

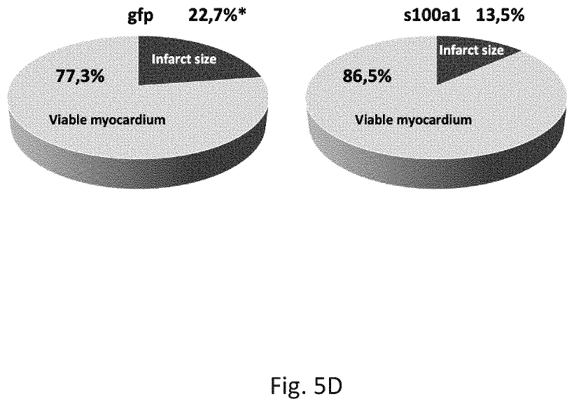

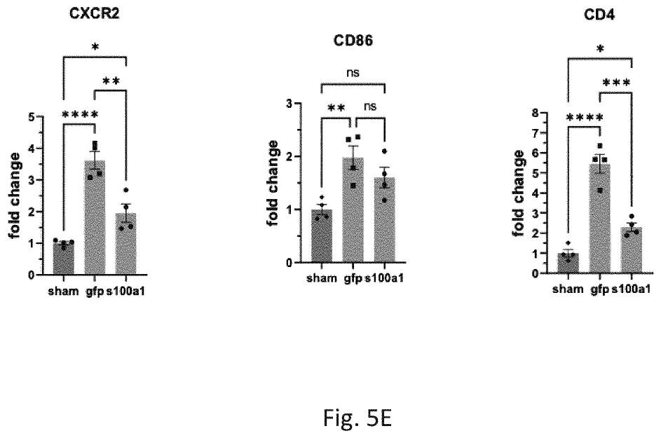

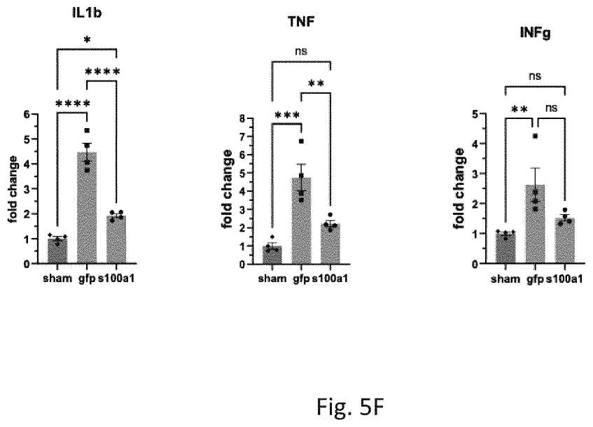

A previously published murine LV-MI model (Most et al. “Cardiac S100A1 protein levels determine contractile performance and propensity toward heart failure after myocardial infarction”, 114, 2006) was employed with the aim to confirm key findings inferred from the signaling pathway analysis in our large animal post-MI model. The experimental set-up is shown in FIG. 5A. Intramyocardial injections were performed immediately after experimental MI. 4-weeks after gene therapy, both LV-EF % and inflammatory gene expression were determined by echocardiography and RT-PCR (black encircled box). Specifically, a total of 2×10¹¹ vgc of rAAV5-hs100a1 and rAAV5-luc were injected into the LV wall of the murine heart (FIG. 5B) prior the ligation of the LAD. Four weeks after treatment, the impact of rAAV5-hs100a1 and controls on post-MI LV performance and the size of MI were determined by echocardiography and immunohistology (J. Takagawa et al., “Myocardial infarct size measurement in the mouse chronic infarction model: comparison of area- and length-based approaches”, H Appl Physiol, 102, 2007), respectively. Since an effect of S100A1 gene therapy on the immune system activity in remodeled and dysfunctional hearts have not yet been described, efforts focused on the myocardial abundance of transcripts of marker genes related to innate and adaptive immune system activity by RT-PCR analysis.

FIG. 5C and FIG. 5D show that rAAV5-hs100a1 treatment improved LV-EF % and mitigated MI size in the murine model compared to the control groups after four weeks. In line with the post-MI large animal model that had received rAAV5-hs100a1, post-MI mice that received an acute rAAV5-hs100a1 intra-myocardial injection exhibited lower levels of various marker genes for myocardial macrophage, neutrophil and T-cell presence and pro-inflammatory cytokines, including e.g., cd68, cxcr2 and cd4 as well as il-1b, tnf and inf-g compared to the control treatment (FIGS. 5E and F).

The Examples show inter alia that S100A1 is an effective, safe and specific therapeutic means to combat myocardial infarct extension.

1. An S100A1 protein, a biologically active fragment or variant thereof or a nucleic acid encoding said S100A1 protein or the biologically active fragment or variant thereof for use in treating and/or preventing infarct extension in a patient in need thereof, wherein the fragment or variant has at least 80% sequence identity to SEQ ID NO:1.

2. A vector comprising a nucleic acid encoding an S100A1 protein or a biologically active fragment or variant thereof having at least 80% sequence identity to SEQ ID NO: 1, for use in treating and/or preventing infarct extension in a patient in need thereof.

3. A pharmaceutical composition comprising
(i) an S100A1 protein, a biologically active fragment or variant thereof having at least 80% sequence identity to SEQ ID NO:1 or a nucleic acid encoding said S100A1 protein, fragment or variant;
(ii) a vector comprising a nucleic acid encoding an S100A1 protein or a biologically active fragment or variant thereof having at least 80% sequence identity to SEQ ID NO: 1; or
(iii) a pharmaceutically acceptable salt of (i) or (ii),
for use in treating and/or preventing infarct extension in a patient in need thereof, optionally wherein the pharmaceutical composition further comprises a pharmaceutically acceptable excipient, carrier, and/or diluent.

4. The S100A1 protein, the biologically active fragment or variant thereof, the nucleic acid, the vector or the pharmaceutical composition for use according to one of claims 1 to 3, wherein the S100A1 protein has the sequence as set forth in SEQ ID NO:1.

5. The vector or the pharmaceutical composition for use according to claim 2, 3 or 4, wherein the vector is selected from the group consisting of plasmid vectors, cosmid vectors, phage vectors such as lambda phage, filamentous phage vectors, viral vectors, viral like particles, and bacterial spores.

6. The vector or the pharmaceutical composition for use according to claim 5, wherein the viral vector is selected from the group consisting of an adenoviral vector, adeno-associated viral (AAV) vector, alphaviral vector, herpes viral vector, measles viral vector, pox viral vector, vesicular stomatitis viral vector, retroviral vector and lentiviral vector.

7. The vector or the pharmaceutical composition for use according to claim 6, wherein the adeno-associated viral (AAV) vector is selected from the group consisting of AAV5, AAV6, and AAV9, preferably AAV5.

8. The vector or the pharmaceutical composition for use according to any one of claims 2 to 7, wherein the expression of the S100A1 protein is controlled by a heart tissue specific promoter.

9. The vector or the pharmaceutical composition for use according to claim 8, wherein the heart-tissue specific promoter is selected from the group consisting of Cardiac Actin Enhancer/Elongation Factor 1 promoter Cytomegalo-virus enhancer/Myosin light chain ventricle 2 promoter and Troponin.

10. The S100A1 protein, the biologically active fragment or variant thereof, the nucleic acid, the vector or the pharmaceutical composition for use according to any one of claims 1 to 9, wherein the protein, the biologically active fragment thereof, the nucleic acid, the vector or the pharmaceutical composition is administered via the oral, intravenous, intramucosal, intraarterial, intramuscular or intracoronal route.

11. The S100A1 protein, the biologically active fragment or variant thereof, the nucleic acid, the vector or the pharmaceutical composition for use according to claim 10, wherein the protein, the biologically active fragment or variant thereof, the nucleic acid, the vector or the pharmaceutical composition is administered via the intravenous route, preferably wherein the protein, the nucleic acid, the vector or the pharmaceutical composition is administered retrograde into the coronary venous system of the patient.

12. The S100A1 protein, the biologically active fragment or variant thereof, the nucleic acid, the vector or the pharmaceutical composition for use according to any one of claims 1 to 11, wherein the protein, the biologically active fragment or variant thereof, the vector or the pharmaceutical composition is administered between 1 day and four weeks after a myocardial infarction in said patient has become post-ischemic.

13. The S100A1 protein, the biologically active fragment or variant thereof, the nucleic acid, the vector or the pharmaceutical composition for use according to any one of claims 1 to 12, wherein the intracellular level of the S100A1 protein is raised for a period of at least 7 days.

14. The vector or the pharmaceutical composition for use according to any one of claims 2 to 13, wherein the vector is administered in a single dose of 1×10¹³ vector genomic copies.

15. Method for treating and/or preventing infarct extension in a patient in need thereof, the method comprising administering to the patient:
(i) an S100A1 protein, a biologically active fragment or variant thereof, or a nucleic acid encoding said S100A1 protein or said fragment or variant thereof,
(ii) a vector comprising a nucleic acid encoding an S100A1 protein or a biologically active fragment thereof, or
(iii) a pharmaceutically acceptable salt of (i) or (ii), wherein the fragment or variant has at least 80% sequence identity to SEQ ID NO:1.

16. The method according to claim 15, wherein the S100A1 protein has the sequence as set forth in SEQ ID NO:1.

17. The method according to claim 15, wherein the vector is selected from the group consisting of plasmid vectors, cosmid vectors, phage vectors such as lambda phage, filamentous phage vectors, viral vectors, viral like particles, and bacterial spores.

18. The method according to claim 17, wherein the viral vector is selected from the group consisting of an adenoviral vector, adeno-associated viral (AAV) vector, alphaviral vector, herpes viral vector, measles viral vector, pox viral vector, vesicular stomatitis viral vector, retroviral vector and lentiviral vector.

19. The method according to claim 18, wherein, wherein the adeno-associated viral (AAV) vector is selected from the group consisting of AAV5, AAV6, and AAV9.

20. The method according to claim 15, wherein the expression of the S100A1 protein is controlled by a heart tissue specific promoter.

21. The method according to claim 20, wherein the heart-tissue specific promoter is selected from the group consisting of Cardiac Actin Enhancer/Elongation Factor 1 promoter Cytomegalo-virus enhancer/Myosin light chain ventricle 2 promoter and Troponin.

22. The method according to claim 15, wherein the protein, the biologically active fragment thereof, the nucleic acid, the vector or the pharmaceutical composition is administered via the oral, intravenous, intramucosal, intraarterial, intramuscular or intracoronal route.

23. The method according to claim 22, wherein the protein, the biologically active fragment thereof, the nucleic acid, the vector or the pharmaceutical composition is administered via the intravenous route, preferably wherein the protein, the nucleic acid, the vector or the pharmaceutical composition is administered retrograde into the coronary venous system of the patient.

24. The method according to claim 15, wherein the protein, the biologically active fragment thereof, the vector or the pharmaceutical composition is administered between 1 day and four weeks after a myocardial infarction in said patient has become post-ischemic.

25. The method according to claim 15, wherein the intracellular level of the S100A1 protein is raised for a period of at least 7 days.

26. The method according to claim 15, wherein the vector is administered in a single dose of 1×10¹³ vector genomic copies (vgc).


SEQUENCE LISTING (1 sequence)

SEQ ID NO: 1 - protein, 94 aa, Homo sapiens
MGSELETAMETLINVFHAHSGKEGDKYKLSKKELKELLQTELSGFLDAQKDVDAVDKVMKELDENGDGEVDFQEYVVLVAALTVACNNFFWENS